(19) United States
(12) Patent Application Publication
(10) Pub. No.: US 2005/0261234 A1
(43) Pub. Date: Nov. 24, 2005

(54) GFAP-based gene therapy for treatment of retinal diseases

(75) Inventors: C. Kathleen Dorey, Boca Raton, FL (US); Janet C. Blanks, Boca Raton, FL (US); Howard Prentice, Boca Raton, FL (US)

Correspondence Address: AKERMAN SENTERFITT, P.O. BOX 3188, WEST PALM BEACH, FL, 33402-3188, US

(73) Assignee: Florida Atlantic University, Boca Raton, FL (US)

(21) Appl. No.: 11/112,254

(22) Filed: Apr. 22, 2005

Related U.S. Application Data
(60) Provisional application No. 60/564,475, filed on Apr. 22, 2004.

(51) Int. Cl. A61K 48/00; A61N 1/30; C07H 21/04; C12N 5/08; C12N 15/09

(52) U.S. Cl. 514/44; 604/20; 435/320.1; 435/366; 536/23.2

(57) ABSTRACT

Compositions and methods for reducing neovascularization. Purified nucleic acid constructs and vectors encoding an anti-angiogenic protein operably linked to a GFAP promoter. Vectors can include at least one hypoxia regulated element, enhancer element and silencer element. Gene therapy methods for reducing, delaying or preventing neovascularization based on the nucleic acid constructs and vectors.

### CROSS-REFERENCE TO RELATED APPLICATIONS

The present application claims the priority of U.S. Provisional Patent application No. 60/564,475 filed Apr. 22, 2004; and entitled “GFAP-BASED GENE THERAPY FOR NEOVASCULARIZATION” which is incorporated herein by reference in its entirety.

### FIELD OF THE INVENTION

This invention relates generally to the fields of medicine and gene therapy. More particularly, the invention relates to nucleic acid molecules, vectors, and methods of using the foregoing compositions to reduce neovascularization.

### BACKGROUND

Neovascularization refers to the formation of new blood vessels. Although such formation, also known as angiogenesis, is part of normal development, wound healing and maintenance of certain adult tissues such as the uterus, neovascularization is a serious complication of many diseases including cancer, rheumatoid arthritis and psoriasis as well as several ophthalmic conditions. Neovascularization is often characterized by vessels exhibiting abnormal fragility and inappropriate localization. In the eye, neovascularization occurs in both diabetic retinopathy and age-related macular degeneration (AMD), with devastating results often leading to blindness. The current treatment for neovascularization in diabetes employs lasers to create a grid of small laser burns in the retina to reduce tissue hypoxia leading to neovascularization. In AMD, lasers are used to close off or eliminate abnormal vessels. These treatments slow loss of vision, and but are palliative only. Recurrence is a common problem, leading to progressive vision loss, and in some cases ultimate blindness.

It has been established in animal models that neovascularization can be effectively inhibited by molecular antagonists of angiogenic factors, such as pigment epithelial derived factor (PEDF), angiostatin, endostatin, and molecules such as Flt1 that block the activity of vascular endothelial growth factor (VEGF) (Dawson, D. W., et al., 1999 Science 285:245-8; 43; Igarashi, T. et al. 2003, Gene Ther, 10:219-26; O'Reilly, M. S. et al.1997 Cell 88:277-85; Raisler, B. J. et al. 2002 Proc Natl Acad Sci USA 99:8909-14; Kuo, C. J., et al. 2001 Proc Natl Acad Sci USA 98:4605-10). Long term delivery of these agents by gene therapy using vectors encoding the gene products is an established alternative to repeated intraocular injection of these factors. For example, it has been shown that a single injection of a recombinant adeno-associated virus (rAAV) or lentiviral vector can result in sustained transgene expression in retinal cells without significant inflammatory response (Ali, R. R. et al. 1998 Gene Ther 5:1561-5; Auricchio, A. et al. 2002 Mol Ther 6:490-4; Bainbridge J. W. et al. 2003 Gene Ther 10:1049-54; Alexander, M. Y. et al. 1999 Clin Exp Pharmacol Physiol 26:661-68).

Despite these advances, long-term inhibition of neovascularization by sustained and unregulated expression of anti-angiogenic proteins presents a risk for adverse effects. For example, indiscriminate expression of anti-angiogenic factors, as delivered by current vectors and protocols, can interfere with the normal physiology and/or development or healing of other tissues, adversely affecting blood vessels critical for normal function, such as in the uterus and brain (Haigh, J. J. et al. 2003 Dev Biol 262:225-41). Even within a tissue of interest, localized delivery of a therapeutic gene product such as an anti-angiogenic agent may be preferable to widespread distribution. Accordingly, there exists a clear need for new gene therapy vectors and methods that can regulate the expression of anti-angiogenic gene products locally within a tissue such as the retina, and/or can selectively regulate their expression, for example in response to adverse conditions in a local tissue environment that are conducive to the formation of these unwanted new vessels.

### SUMMARY

The invention relates to isolated nucleic acid constructs, vectors and gene therapy methods that suppress or prevent recurrence of neovascularization. The various embodiments of the invention utilize a promoter from the glial fibrillary acidic protein gene (GFAP) to drive expression of anti-angiogenic factors in transduced cells. The construct and vector design is advantageous for selective delivery of therapeutic gene products to regions of tissue injury, hypoxia or other forms of stress because GFAP is upregulated in GFAP-expressing cells in the nervous system in response to such stressful physiological conditions. Accordingly, the vectors direct expression of therapeutic genes in a targeted manner, delivering enhanced and/or regionally localized transgene expression to those tissue sites most in need. Advantageously, generalized expression of the anti-angiogenic agents is avoided at sites where this is not indicated or desirable.

Accordingly, in one aspect, the invention includes a purified nucleic acid construct comprising: at least one nucleic acid encoding an anti-angiogenic protein, operably linked to a GFAP promoter. The nucleic acid can further include at least one of a hypoxia regulated element, an enhancer element, and a silencer element.

In preferred embodiments, the anti-angiogenic protein included in the nucleic acid construct is endostatin, angiostatin or Tubedown-1.

Another aspect of the invention is a vector comprising: a purified nucleic acid construct including at least one nucleic acid encoding an anti-angiogenic protein operably linked to a GFAP promoter. The anti-angiogenic protein can be endostatin, angiostatin or Tubedown-1.

The vector can be an expression vector. The expression vector can be a plasmid. The vector can also be a viral vector such as an adenoviral vector, a rAAV vector or a lentiviral vector.

Yet another embodiment of the invention is a cell transduced with the above-described vectors.

Yet a further aspect of the invention is a method of preventing, reducing or delaying neovascularization. The method includes the steps of: (a) providing a subject having or at risk of developing neovascularization in a tissue; and (b) transducing at least one GFAP-expressing cell type within the tissue of the subject with an expression vector including a purified nucleic acid construct including at least one nucleic acid encoding an anti-angiogenic protein operably linked to a GFAP promoter. Expression of the factor by the transduced cell prevents, reduces or delays neovascularization in the tissue of the subject.

This tissue targeted by the method can be an ocular tissue selected from at least one of retina, vitreous and choroid.

In preferred embodiments, the anti-angiogenic protein expressed in the method can be angiostatin, endostatin or Tubedown-1.

In some versions of the method, the expression vector can further include a hypoxia-regulated element. Upon use of such vectors, expression of the anti-angiogenic protein by the cell is increased under hypoxic conditions.

Yet a further variation of the gene therapy method includes an additional step (c) of: irradiating the tissue with a laser beam, radiation, Cystatin C, transpupillary thermography or any other form of light energy in an amount sufficient to upregulate GFAP promoter expression in the GFAP-expressing cell type. As used herein, “an amount sufficient” to upregulate, activate refers to light, or photons at a wavelength of from about 10 nm up to 2000 nm. Usually the wavelength is between about 500 nm to about 800 nm. The level of expression of the anti-angiogenic protein driven by the GFAP promoter is increased in an amount sufficient to prevent, reduce or delay neovascularization in the transfected tissue.

A variation of the method for ophthalmic applications is a method of preventing, reducing or delaying neovascularization in an eye, including the steps of :(a) providing a subject having or at risk of developing a condition involving neovascularization in a compartment of at least one eye; and (b) transducing at least one GFAP-expressing cell type in an eye of the subject with an expression vector including at least one nucleic acid encoding an anti-angiogenic protein operably linked to a GFAP promoter, wherein expression of the anti-angiogenic protein by the transduced cell prevents, reduces or delays neovascularization in the eye of the subject.

The GFAP-expressing cell type targeted by the ocular gene therapy method can be a Muller cell or a retinal glial cell.

Yet a further variation of the ocular gene therapy method includes the additional step (c) of irradiating the tissue with a laser beam sufficient to upregulate GFAP promoter expression in the GFAP-expressing cell type, wherein the level of expression of the anti-angiogenic protein driven by the GFAP promoter is increased in an amount sufficient to prevent, reduce or delay neovascularization in the tissue. The tissue within an eye can include vitreous, retina and choroid. The light or energy can be a laser beam, radiation, Cystatin C, transpupillary thermography or any other form of light energy in an amount sufficient to upregulate GFAP promoter expression in the GFAP-expressing cell type. Light, or photons are at a wavelength of from about 10 nm up to 2000 nm. Usually the wavelength is between about 500 nm to about 800 nm.

In a preferred embodiment, a vector comprises a purified nucleic acid construct comprising: at least one nucleic acid encoding an anti-angiogenic protein operably linked to a GFAP promoter. The anti-angiogenic protein is selected from the group consisting of endostatin, angiostatin and Tubedown-1. Preferably, the vector is a viral vector selected from the group consisting of an adenoviral vector, a rAAV vector and a lentiviral vector. Gene expression is regulated by a GFAP or HSP promoter which is up-regulated by irradiating tissue with a laser beam, light, radiation, Cystatin C, transpupillary thermography in an amount sufficient to upregulate GFAP promoter driven expression of a nucleic acid under said promoters control. The amount of light energy sufficient to upregulate GFAP promoter driven expression of a nucleic acid under said promoters control is a wavelength of light energy is from about 10 nm up to 2000 nm. Preferably, upregulation of the GFAP promoter upregulates expression of the anti-angiogenic factor as compared to a normal tissue and a tissue comprising said vector.

In another preferred embodiment, a cell transduced with a vector comprising: at least one nucleic acid encoding an anti-angiogenic protein, operably linked to a GFAP promoter; at least one of a hypoxia regulated element, an enhancer element and a silencer element; anti-angiogenic protein is selected from the group consisting of endostatin, angiostatin and Tubedown-1. The GFAP promoter is up-regulated by by irradiating tissue comprising the vector with a laser beam, light, radiation, Cystatin C, transpupillary thermography in an amount sufficient to upregulate GFAP promoter driven expression of a nucleic acid under said promoters control.

Unless otherwise defined, all technical terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference for the proposition cited. In the case of conflict, the present specification, including any definitions will control. The particular embodiments discussed below are illustrative only and not intended to be limiting.

Other features and advantages of the invention will be apparent from the following detailed description, and from the claims.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIG. 1 is a schematic diagram showing a design for an anti-angiogenic gene therapy vector, according to an embodiment of the invention.

FIG. 2A-2D are photographs of a histological stain showing that the doxicyclin had no effect on the retinas of 6 week old mice when the tubedown-1 was regulated only by tetracycline responsive elements. However, in animals with endothelial cell specific, dox-induced tubedown antisense (ASTBDN-1), two weeks exposure to dox resulted in large blood vessels along the vitreal surface (V) of the neural retina (NR) (FIG. 2B). These vessels developed into larger distended vessels (FIG. 2C) and vascular complexes (FIG. 2D) after 10 or 12 weeks of DOX exposure. Arrows in each image identify profiles of vessels (FIG. 2A and FIG. 2B) or segments of dilated vessels (FIG. 2C and FIG. 2D). Tubedown-1 acts as a negative regulator of vascular growth in vivo.

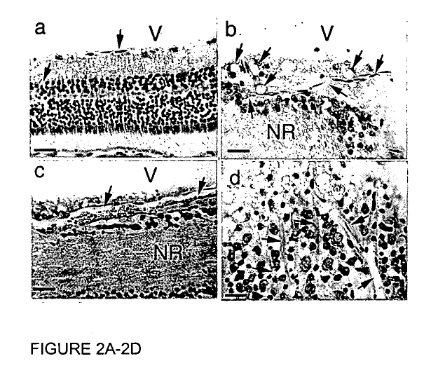

### DETAILED DESCRIPTION

The invention includes purified nucleic acid constructs, vectors containing these constructs, and gene therapy methods that utilize a promoter derived from glial fibrillary acidic protein (GFAP) to drive expression of anti-angiogenic proteins in transduced cells. The construct and vector design is advantageous for selective delivery of therapeutic gene products to regions of tissue injury, hypoxia or other forms of stress because GFAP is upregulated in GFAP-expressing cells in the nervous system in response to such stressful physiological conditions. Accordingly, the vectors drive expression of therapeutic genes in a targeted manner, delivering enhanced and/or regionally localized transgene expression to those tissue sites most in need, thereby avoiding generalized expression of the anti-angiogenic agents where such expression is not indicated or desirable.

General Molecular Biology

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

Definitions

The present section provides definitions of the terms used in the present invention in order to facilitate a better understanding of the invention.

As used herein, the singular forms “a”, “an” and “the” include plural referents unless the context clearly dictates otherwise.

A “retinopathy” is defined generally as any disease of the retina. Briefly, diabetic retinopathy is one of the most common complications of diabetes as a result of non-resolving vitreous hemorrhage, traction retinal detachment or diabetic maculopathy. All of these are consequences of retinal capillary closure or leakage. “Sight-threatening diabetic retinopathy” refers to diabetic complications affecting the retina that predictably lead to severe loss of vision. Retinal disease is one of several complications of diabetes and is primarily the result of disruption of small blood vessels and the uncontrolled growth of new vessels. In some situations, these changes affect the macula, which is the area of the retina that is specialized to color and visual acuity.

By “propensity,” “predisposition” or “susceptibility” for disease what is meant is that certain alleles are “associated” with sight-threatening diabetic retinopathy. They are thus over represented in frequency in individuals with disease as compared to healthy individuals.

The terms “amino acid” or “amino acid sequence,” as used herein, refer to an oligopeptide, peptide, polypeptide, or protein sequence, or a fragment of any of these, and to naturally occurring or synthetic molecules. Where “amino acid sequence” is recited herein to refer to an amino acid sequence of a naturally occurring protein molecule, “amino acid sequence” and like terms are not meant to limit the amino acid sequence to the complete native amino acid sequence associated with the recited protein molecule.

As used herein, the term “angiogenesis” means the generation of new blood vessels into a tissue or organ, and involves endothelial cell proliferation. Under normal physiological conditions, humans or animals undergo angiogenesis only in very specific restricted situations. For example, angiogenesis is normally observed in wound healing, fetal and embryonal development, and formation of the corpus luteum, endometrium and placenta. The term “endothelium” means a thin layer of flat epithelial cells that lines serous cavities, lymph vessels, and blood vessels. “Anti-angiogenic activity” therefore refers to the capability of a composition to inhibit the growth of blood vessels. The growth of blood vessels is a complex series of events, and includes localized breakdown of the basement membrane lying under the individual endothelial cells, proliferation of those cells, migration of the cells to the location of the future blood vessel, reorganization of the cells to form a new vessel membrane, cessation of endothelial cell proliferation, and, incorporation of pericytes and other cells that support the new blood vessel wall. “Anti-angiogenic activity” as used herein therefore includes interruption of any or all of these stages, with the end result that formation of new blood vessels is inhibited.

An “anti-angiogenic” factor is a molecule that inhibits angiogenesis, particularly by blocking endothelial cell migration, endothelial cell activation, intervention in cell attachment and cell proliferation, production of VEGF receptors, interference with interaction of growth factors and their receptors, inhibit essential proteases, promote endothelial cell death, tissue invasion and/or assembly into tubules, etc.. Such factors include fragments of angiogenic proteins that are inhibitory (such as the ATF of urokinase), angiogenesis inhibitory factors, such as angiostatin and endostatin; and soluble receptors of angiogenic factors, such as the urokinase receptor or FGF/VEGF receptor. The term “angiostatin”, which is derived from the amino-terminal fragment of plasinogen, includes the anti-angiogenic fragment of angiostatin having kringles 1 to 3. Generally, an anti-angiogenic factor for use in the invention is a protein or polypeptide encoded by a gene transfected into tumors using the vectors of the invention.

A “variant” of a polypeptide or protein is any analogue, fragment, derivative, or mutant which is derived from a polypeptide or protein and which retains at least one biological property of the polypeptide or protein. Different variants of the polypeptide or protein may exist in nature. These variants may be allelic variations characterized by differences in the nucleotide sequences of the structural gene coding for the protein, or may involve differential splicing or post-translational modification. The skilled artisan can produce variants having single or multiple amino acid substitutions, deletions, additions, or replacements. These variants may include, inter alia: (a) variants in which one or more amino acid residues are substituted with conservative or non-conservative amino acids, (b) variants in which one or more amino acids are added to the polypeptide or protein, (c) variants in which one or more of the amino acids includes a substituent group, and (d) variants in which the polypeptide or protein is fused with another polypeptide such as serum albumin. The techniques for obtaining these variants, including genetic (suppressions, deletions, mutations, etc.), chemical, and enzymatic techniques, are known to persons having ordinary skill in the art. If such allelic variations, analogues, fragments, derivatives, mutants, and modifications, including alternative mRNA splicing forms and alternative post-translational modification forms result in derivatives of the polypeptide which retain any of the biological properties of the polypeptide, they are intended to be included within the scope of this invention.

The term “anti-angiogenic activity” as used herein, refers to the inhibition and/or moderation of angiogenesis.

The term “angiogenesis-associated disease” is used herein, for purposes of the specification and claims, to mean certain pathological processes in humans where angiogenesis is abnormally prolonged. Such angiogenesis-associated diseases include diabetic retinopathy, chronic inflammatory diseases, rheumatoid arthritis, dermatitis, psoriasis, stomach ulcers, and most types of human solid tumors.

The term “angiogenesis inhibitor” is used herein, to mean a biomolecule including, but not limited to, peptides, proteins, enzymes, polysaccharides, oligonucleotides, DNA, RNA, recombinant vectors, and drugs which function to inhibit angiogenesis. Angiogenesis inhibitors are known in the art and include natural and synthetic biomolecules.

The term “anti-angiogenic therapy” is used herein, for purposes of the specification and claims, to mean therapy directed against angiogenesis (i.e., the formation of new capillary blood vessels leading to neovascularization), and/or existing vasculature and relating to a disease condition (e.g., vascular targeting therapy).

As used herein, the term “biologically active” refers to a protein having structural, regulatory, or biochemical functions of a naturally occurring molecule.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “derivative,” as used herein, refers to the chemical modification of a polypeptide sequence, or a polynucleotide sequence. Chemical modifications of a polynucleotide sequence can include, for example, replacement of hydrogen by an alkyl, acyl, or amino group. A derivative polynucleotide encodes a polypeptide which retains at least one biological function of the natural molecule. A derivative polypeptide is one modified, for instance by glycosylation, or any other process which retains at least one biological function of the polypeptide from which it was derived.

As used herein, the term “transfection” means the introduction of a nucleic acid, e.g., an expression vector, into a recipient cell by nucleic acid-mediated gene transfer.

“Transformation”, as used herein, refers to a process in which a cell's genotype is changed as a result of the cellular uptake of exogenous DNA or RNA, and, for example, the transformed cell expresses for example, angiostatin.

As used herein, the term “vector” refers to a nucleic acid molecule capable of transporting another nucleic acid. Vectors capable of directing the expression of genes to which they are operatively linked are referred to herein as “expression vectors”. In general, expression vectors of utility in recombinant DNA techniques are often in the form of “plasmids” which refer to circular double stranded DNA loops which, in their vector form are not bound to the chromosome. Vectors may also be viral vectors wherein the viral vector is selected from the group consisting of a lentivirus, adenovirus, adeno-associated virus and virus-like vectors. The vector may also be a lipid vesicle or liposome wherein the DNA is surrounded by a lipid emulsion that is taken up by the cell. The invention is intended to include such other forms of expression vectors which serve equivalent functions and which become known in the art subsequently hereto. An “expression vector” is a vector capable of expressing a DNA (or cDNA) or RNA molecules cloned into the vector and, in certain cases, producing a polypeptide or protein. Appropriate transcriptional and/or translational control sequences are included in the vector to allow it to be expressed in a cell. Expression of the cloned sequences occurs when the expression vector is introduced into an appropriate host cell. If a eukaryotic expression vector is employed, then the appropriate host cell would be any eukaryotic cell capable of expressing the cloned sequences. Vectors include chemical conjugates such as described in WO 93/04701, which has a targeting moiety (e.g. a ligand to a cellular surface receptor), and a nucleic acid binding moiety (e.g. polylysine), viral vector (e.g. a DNA or RNA viral vector), fusion proteins such as described in PCT/US95/02140 (WO 95/22618) which is a fusion protein containing a target moiety (e.g. an antibody specific for a target cell) and a nucleic acid binding moiety (e.g. a protamine), plasmids, phage etc. The vectors can be chromosomal, non-chromosomal or synthetic. Preferred vectors include viral vectors, fusion proteins and chemical conjugates. Retroviral vectors include Moloney murine leukemia viruses. DNA viral vectors are preferred. Viral vectors can be chosen to introduce the cytokine or chemokine to cells of choice. Such vectors include pox vectors such as orthopox or avipox vectors, herpesvirus vectors such as herpes simplex I virus (HSV) vector (Geller, A. I et al, J. Neurochem., 64:487(1995); Lim, F., et al., in DNA Cloning: Mammalian Systems, D. Glover, Ed. (Oxford Univ. Press, Oxford, England) (1995); Geller, A. I. et al., Proc. Natl. Acad. Sci. USA 87:1149 (1990)) Adenovirus vectors (LeGal LaSalle et al., Science, 259:988 (1993); Davidson, et al., Nat. Genet. 3:219 (1993); Yang et al., J. Virol. 69:2004 (1995)) and Adeno-associated virus vectors (Kaplitt, M. G. et al., Nat. Genet. 8:148 (1994)). Pox viral vectors introduce the gene into the cells cytoplasm. Avipox virus vectors result in only short term expression of the nucleic acid. Adenovirus vectors, adeno-associated virus vectors and herpes simplex virus vectors are preferred for introducing the nucleic acid into neural cells. The adenovirus vector results in a shorter term expression (about 2 months) than adeno-associated virus (about 4 months), which in turn is shorter than HSV vectors. The vectors can be introduced by standard techniques, e.g. infection, transfection, transduction or transformation. Examples of modes of gene transfer include for example, naked DNA calcium phosphate precipitation, DEAE dextran, electroporation, protoplast fusion, lipofection, cell microinjection and viral vectors.

A nucleic acid molecule, such as DNA, RNA, is said to be “capable of expressing” a polypeptide if it contains nucleotide sequences which contain transcriptional and translational regulatory information and such sequences are “operably linked” to a nucleotide sequence that encodes the polypeptide. An operable linkage is a linkage in which the regulatory DNA sequences and the DNA sequence sought to be expressed are connected in such a way as to permit gene expression. Regulatory elements include elements such as a promoter, an initiation codon, a stop codon and a polyadenylation signal.

Vectors can be constructed which also comprise a detectable/selectable marker gene. In preferred embodiments these marker genes are fluorescent proteins such as green fluorescent protein (GFP), cyan—(CFP), yellow—(YFG), blue—(BFP), red—(RFP) fluorescent proteins; enhanced green fluorescent protein (EGFP), EYFP, EBFP, Nile Red, dsRed, mutated, modified, or enhanced forms thereof, and the like.

As used herein, the “green-fluorescence protein” is a gene construct which in transfected or infected cells, respectively, shines green under ultraviolet light and thus enables the detection of a cell transfected or infected, respectively, with GFP in a simple manner.

Uses of green fluorescent protein for the study of gene expression and protein localization are well known. The compact structure makes GFP very stable under diverse and/or harsh conditions such as protease treatment, making GFP an extremely useful reporter in general.

New versions of green fluorescent protein have been developed, such as a “humanized” GFP DNA, the protein product of which has increased synthesis in mammalian cells. One such humanized protein is “enhanced green fluorescent protein” (EGFP). Other mutations to green fluorescent protein have resulted in blue-, cyan- and yellow-green light emitting versions.

Endogenously fluorescent proteins have been isolated and cloned from a number of marine species including the sea pansies Renilla reniformris, R. kollikeri and R. mullerei and from the sea pens Ptilosarcus, Stylatula and Acanthoptilum, as well as from the Pacific Northwest jellyfish, Aequorea victoria; Szent-Gyorgyi et al. (SPIE conference 1999), D. C. Prasher et al., Gene, 111:229-233 (1992) and several species of coral (Matz et al. Nature Biotechnology, 17 969-973 (1999). These proteins are capable of forming a highly fluorescent, intrinsic chromophore through the cyclization and oxidation of internal amino acids within the protein that can be spectrally resolved from weakly fluorescent amino acids such as tryptophan and tyrosine.

“Regulatory region” means a nucleic acid sequence which regulates the expression of a second nucleic acid sequence. A regulatory region may include sequences which are naturally responsible for expressing a particular nucleic acid (a homologous region) or may include sequences of a different origin (responsible for expressing different proteins or even synthetic proteins). In particular, the sequences can be sequences of eukaryotic or viral genes or derived sequences which stimulate or repress transcription of a gene in a specific or non-specific manner and in an inducible or non-inducible manner. Regulatory regions include origins of replication, RNA splice sites, enhancers, transcriptional termination sequences, signal sequences which direct the polypeptide into the secretory pathways of the target cell, and promoters.

A regulatory region from a “heterologous source” is a regulatory region which is not naturally associated with the expressed nucleic acid. Included among the heterologous regulatory regions are regulatory regions from a different species, regulatory regions from a different gene, hybrid regulatory sequences, and regulatory sequences which do not occur in nature, but which are designed by one having ordinary skill in the art.

A “cassette” refers to a segment of DNA that can be inserted into a vector at specific restriction sites. The segment of DNA encodes a polypeptide of interest, and in the case of coding sequences, the cassette and sites of insertion are chosen to ensure insertion of the coding sequences in the proper reading frame for transcription and translation.

“Heterologous” DNA refers to DNA not naturally located in the cell, or in a chromosomal site of the cell. Preferably, the heterologous DNA includes a gene foreign to the cell.

A “nucleic acid” is a polymeric compound comprised of covalently linked subunits called nucleotides. Nucleic acid includes polyribonucleic acid (RNA) and polydeoxyribonucleic acid (DNA), both of which may be single-stranded or double-stranded. DNA includes cDNA, genomic DNA, synthetic DNA, and semi-synthetic DNA. The sequence of nucleotides or nucleic acid sequence that encodes a protein is called the sense sequence. A “recombinant DNA molecule” is a DNA molecule that has undergone a molecular biological manipulation.

A DNA “coding sequence” is a double-stranded DNA sequence which is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. Alternative 5′ terminae are also possible for certain genes. A polyadenylation signal and transcription termination sequence will usually be located 3′ to the coding sequence. Transcriptional and translational control sequences are DNA regulatory sequences, such as promoters, enhancers, terminators, and the like, that provide for the expression of a coding sequence in a host cell. In eukaryotic cells, polyadenylation signals are control sequences.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then optionally trans-RNA spliced and translated into the protein encoded by the coding sequence.

A “signal sequence” is included at the beginning of the coding sequence of a protein to be expressed on the surface of a cell. This sequence encodes a signal peptide, N-terminal to the mature polypeptide, that directs the host cell to translocate the polypeptide. The term “translocation signal sequence” is used herein to refer to this sort of signal sequence. Translocation signal sequences can be found associated with a variety of proteins native to eukaryotes and prokaryotes, and are often functional in both types of organisms.

The term “corresponding to” is used herein to refer similar or homologous sequences, whether the exact position is identical or different from the molecule to which the similarity or homology is measured. A nucleic acid or amino acid sequence alignment may include spaces. Thus, the term “corresponding to” refers to the sequence similarity, and not the numbering of the amino acid residues or nucleotide bases.

As used herein, the term “oligonucleotide specific for” refers to an oligonucleotide having a sequence (i) capable of forming a stable complex with a portion of the targeted gene, or (ii) capable of forming a stable duplex with a portion of a mRNA transcript of the targeted gene.

As used herein, the terms “oligonucleotide”, “siRNA” “siRNA oligonucleotide” and “siRNA's” are used interchangeably throughout the specification and include linear or circular oligomers of natural and/or modified monomers or linkages, including deoxyribonucleosides, ribonucleosides, substituted and alpha-anomeric forms thereof, peptide nucleic acids (PNA), ed nucleic acids (LNA), phosphorthiorate, methylphosphonate, and the like. Oligonucleotides are capable of specifically binding to a target polynucleotide by way of a regular pattern of monomer-to-monomer interactions, such as Watson-Crick type of base pairing, Hoögsteen or reverse Hoögsteen types of base pairing, or the like.

The oligonucleotide may be “chimeric”, that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide in the case of an oligonucleotide compound. These oligonucleotides typically comprise at least one region wherein the oligonucleotide is modified in order to exhibit one or more desired properties. The desired properties of the oligonucleotide include, but are not limited, for example, to increased resistance to nuclease degradation, increased cellular uptake, and/or increased binding affinity for the target nucleic acid. Different regions of the oligonucleotide may therefore have different properties. The chimeric oligonucleotides of the present invention can be formed as mixed structures of two or more oligonucleotides, modified oligonucleotides, oligonucleosides and/or oligonucleotide analogs as described above.

The oligonucleotide can be composed of regions that can be linked in “register”, that is, when the monomers are linked consecutively, as in native DNA, or linked via spacers. The spacers are intended to constitute a covalent “bridge” between the regions and have in preferred cases a length not exceeding about 100 carbon atoms. The spacers may carry different functionalities, for example, having positive or negative charge, carry special nucleic acid binding properties (intercalators, groove binders, toxins, fluorophors etc.), being lipophilic, inducing special secondary structures like, for example, alanine containing peptides that induce alpha-helices.

As used herein, the term “monomers” typically indicates monomers linked by phosphodiester bonds or analogs thereof to form oligonucleotides ranging in size from a few monomeric units, e.g., from about 3-4, to about several hundreds of monomeric units. Analogs of phosphodiester linkages include: phosphorothioate, phosphorodithioate, methylphosphomates, phosphoroselenoate, phosphoramidate, and the like, as more fully described below.

In the present context, the terms “nucleobase” covers naturally occurring nucleobases as well as non-naturally occurring nucleobases. It should be clear to the person skilled in the art that various nucleobases which previously have been considered “non-naturally occurring” have subsequently been found in nature. Thus, “nucleobase” includes not only the known purine and pyrimidine heterocycles, but also heterocyclic analogues and tautomers thereof. Illustrative examples of nucleobases are adenine, guanine, thymine, cytosine, uracil, purine, xanthine, diaminopurine, 8-oxo-N⁶-methyladenine, 7-deazaxanthine, 7-deazaguanine, N⁴,N⁴-ethanocytosin, N^{6,}N⁶-ethano-2,6-diaminopurine, 5-methylcytosine, 5-(C³-C⁶)-alkynylcytosine, 5-fluorouracil, 5-bromouracil, pseudoisocytosine, 2-hydroxy-5-methyl-4-triazolopyridin, isocytosine, isoguanin, inosine and the “non-naturally occurring” nucleobases described in Benner et al., U.S. Pat. No. 5,432,272. The term “nucleobase” is intended to cover every and all of these examples as well as analogues and tautomers thereof. Especially interesting nucleobases are adenine, guanine, thymine, cytosine, and uracil, which are considered as the naturally occurring nucleobases in relation to therapeutic and diagnostic application in humans.

As used herein, “nucleoside” includes the natural nucleosides, including 2′-deoxy and 2′-hydroxyl forms, e.g., as described in Komberg and Baker, DNA Replication, 2nd Ed. (Freeman, San Francisco, 1992).

“Analogs” in reference to nucleosides includes synthetic nucleosides having modified base moieties and/or modified sugar moieties, e.g., described generally by Scheit, Nucleotide Analogs, John Wiley, New York, 1980; Freier & Altmann, Nucl. Acid. Res., 1997, 25(22), 4429-4443, Toulme, J. J., Nature Biotechnology 19:17-18 (2001); Manoharan M., Biochemica et Biophysica Acta 1489:117-139(1999); Freier S., M., Nucleic Acid Research, 25:4429-4443 (1997), Uhlman, E., Drug Discovery & Development, 3: 203-213 (2000), Herdewin P., Antisense & Nucleic Acid Drug Dev., 10:297-310 (2000), ); 2′-O, 3′-C-linked [3.2.0] bicycloarabinonucleosides (see e.g. N. K Christiensen., et al, J. Am. Chem. Soc., 120: 5458-5463 (1998). Such analogs include synthetic nucleosides designed to enhance binding properties, e.g., duplex or triplex stability, specificity, or the like.

The term “stability” in reference to duplex or triplex formation generally designates how tightly an antisense oligonucleotide binds to its intended target sequence; more particularly, “stability” designates the free energy of formation of the duplex or triplex under physiological conditions. Melting temperature under a standard set of conditions, e.g., as described below, is a convenient measure of duplex and/or triplex stability. Preferably, oligonucleotides of the invention are selected that have melting temperatures of at least 45° C. when measured in 100 mM NaCl, 0.1 mM EDTA and 10 mM phosphate buffer aqueous solution, pH 7.0 at a strand concentration of both the oligonucleotide and the target nucleic acid of 1.5 μM. Thus, when used under physiological conditions, duplex or triplex formation will be substantially favored over the state in which the antigen and its target are dissociated. It is understood that a stable duplex or triplex may in some embodiments include mismatches between base pairs and/or among base triplets in the case of triplexes. Preferably, modified oligonucleotides, e.g. comprising LNA units, of the invention form perfectly matched duplexes and/or triplexes with their target nucleic acids.

As used herein, the term “downstream” when used in reference to a direction along a nucleotide sequence means in the direction from the 5′ to the 3′ end. Similarly, the term “upstream” means in the direction from the 3′ to the 5′ end.

As used herein, the term “gene” means the gene and all currently known variants thereof and any further variants which may be elucidated.

“Diseases and conditions associated with diabetes mellitus” as defined in this application comprise, but are not restricted to hyperglycemia, hyperinsulinaemia, hyperlipidaemia, insulin resistance, impaired glucose metabolism, obesity, diabetic retinopathy, macular degeneration, cataracts, diabetic nephropathy, glomerulosclerosis, diabetic neuropathy, erectile dysfunction, premenstrual syndrome, vascular restenosis and ulcerative colitis. Furthermore, “diseases and conditions associated with diabetes mellitus” comprise, but are not restricted to: coronary heart disease, hypertension, angina pectoris, myocardial infarction, stroke, skin and connective tissue disorders, foot ulcerations, metabolic acidosis, arthritis, osteoporosis and in particular conditions of impaired glucose tolerance.

The term “prevention” means prophylactic administration of the compositions of the invention to healthy patients to prevent the outbreak of the diseases and conditions mentioned herein. Moreover, the term “prevention” means prophylactic administration of such compositions to patients being in a pre-stage of the disease, especially diabetes, to be treated. The term “delay of progression” used herein means administration of the combination to patients being in a pre-stage of the disease, especially diabetes, to be treated in which patients a pre-form of the corresponding disease is diagnosed. The term “method of treating” used herein includes a method of prevention of a disease, i.e. the prophylactic administration of the compositions to healthy patients to prevent the outbreak of the diseases and conditions mentioned herein.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the term “safe and effective amount” or “therapeutic amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives“). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

“Treatment” is an intervention performed with the intention of preventing the development or altering the pathology or symptoms of a disorder. Accordingly, “treatment” refers to both therapeutic treatment and prophylactic or preventative measures. Those in need of treatment include those already with the disorder as well as those in which the disorder is to be prevented.

The term “subject,” or “patient” as used herein, means a human or non-human animal, including but not limited to mammals such as a dog, cat, horse, cow, pig, sheep, goat, primate, rat, and mouse.

As used herein, the term “administering a molecule to a cell” (e.g., an expression vector, nucleic acid, a angiogenic factor, a delivery vehicle, agent, and the like) refers to transducing, transfecting, microinjecting, electroporating, or shooting, the cell with the molecule. In some aspects, molecules are introduced into a target cell by contacting the target cell with a delivery cell (e.g., by cell fusion or by lysing the delivery cell when it is in proximity to the target cell).

The term “ischemic damage” refers to a reduction in the biological capability of a neuronal cell, including cell death, induced by a reduced blood flow, or an otherwise reduced level of oxygen to the affected neuronal cells, whether it be the result of ischemic stroke, hemmorrhagic stroke, hypoxia or the like.

GFAP Promoter-Driven Transgene Expression for Gene Therapy

Ocular disorders which may be treated by the compositions of the invention include proliferative vitreoretinopathy; as well as ocular disorders associated with neovascularization; including diabetic retinopathy, retinopathy of prematurity, age-related macular degeneration, and neovascular glaucoma; and epithelial cell proliferation such as lens capsule epithelial cell proliferation. The use of a GFAP promoter in the nucleic acids, vectors and methods of the invention provides the advantage of restricting expression of the transgene only to GFAP-expressing cell types. GFAP is selectively expressed in certain glial cell types of the nervous system, including astrocytes in the brain, nonmyelinating Schwann cells, Bergmann glia, radial glia and Muller cells and certain other glia of the retina. Accordingly, isolated GFAP promoters are useful for cell-type specific expression in those cell types that express GFAP.

Furthermore, use of a GFAP promoter for gene therapy in the eye is particularly advantageous for selectively driving expression of a transgene in a diseased or damaged retina because, in contrast to the situation in the brain, expression of GFAP under normal circumstances is very low or nonexistent in the retina. Significantly, however, GFAP is strongly upregulated in Muller cells in response to stresses such as ischemia, trauma, retinal detachment and photoreceptor degeneration (reviewed in Sarthy, V. and Ripps, H., The Retinal Muller Cell: Structure and Function, Kluwer Academic/Plenum Press, New York, 2001). Accordingly, a GFAP promoter can be used to selectively deliver a gene therapeutic agent to a tissue in the eye, such as the retina, that is distressed and in need of treatment with the therapeutic agent. In a particularly preferred embodiment, the vectors of the invention can be used to control neovascularization in the eye, for example in the retina by driving expression of anti-angiogenic proteins in Muller cells, and any other GFAP-expressing glial cells of the retina.

A further advantage of targeting Muller cells for expression of the transgene relates to the structure and physical location of these cells within the retina. Muller cells are known to span the width of the retina, extending from the innermost surface of the retina (facing the vitreous) to the outermost portion of the retina (outer limiting membrane) adjacent to the photoreceptor inner segments. By virtue of their unique and expansive localization in the retina, Muller cells have access not only to sites of neovascularization throughout the thickness of the retina itself, but also to sites of neovascularization both on the inner vitreal surface of the retina, and on its the outer surface facing the retinal pigment epithelium (RPE) and choroid. Thus therapeutic anti-angiogenic factors secreted by Muller cells have the potential to target neovascular foci at multiple, widespread sites within the eye.

Immunization Routes

The ocular route is the preferred route of inoculation; however, this designation as the preferred inoculation route is not meant to preclude any other route of administration. Preferred routes of inoculation of the vector are via intravitreal or subretinal routes. The ocular route includes but is not limited to subconjunctival injection, surface drops, a slow-release device such as a collagen shield, a hydrogel contact lens or an ALZA “Ocusert.”

Subconjunctival vaccination is done using proparacaine for anesthesia prior to the injection of 0.2-0.5 ml of vector, in a dose of 10-1000 .μg/inoculation, given in an insulin syringe and a small gauge needle. The injection is given in the lower cul-de-sac ensuring that the vaccine material remains subconjunctival and does not leak out.

The surface drops inoculation involves placing the vector with or without adjuvant in the conjunctival cul-de-sac and then rubbing the eye gently for 30 seconds while held closed. The procedure can be repeated two or three times a day for five days to prolong the exposure, all of which comprise a single vaccination. For better retention, the tear drainage ducts may be temporarily blocked using collagen or other devices. Alternatively, the vector and/or naked DNA may be encapsulated in a microcapsule and then implanted into the eye to facilitate continuous release.

Nucleic Acid Constructs Encoding GFAP-Driven Anti-Angiogenic Proteins

In accordance with the above considerations, one aspect of the invention is a purified nucleic acid construct including at least one nucleic acid encoding an anti-angiogenic protein, operably linked to a GFAP promoter. Any isolated nucleic acid including a GFAP promoter sequence sufficient to drive expression of a transgene of interest in a cell that expresses GFAP can be used in the invention. Methods for isolating GFAP promoter constructs and testing of cell type-specific expression are well known in the art, and generally involve isolation of GFAP gene fragments of selected sizes, including full length genes along with 5′ and 3′ flanking regions, cloning into a suitable expression vector containing a reporter gene, and analysis of cell-specific expression by transfection into cultured cells or retinal explants in vitro, or testing in transgenic animals.

Several exemplary GFAP promoters derived from mouse and human GFAP sequences are known to be useful for transgene expression in astrocytes in mouse brain (Mucke, L M et al., New Biol 3, 465-474, 1991; Galou, M, et al Glia 12, 281-293,1994; Brenner, M et al., J Neurosci 14,1030-1037, 1994). A GFAP promoter that drives Muller cell-specific expression in the retinas of transgenic mice has also been described (Kuzmanovic M et al., Invest. Ophthalmol. Vis. Sci. 44:3606-3613, 2003). Further details of construction of GFAP promoter sequences of use in the invention are provided in examples below.

In another preferred embodiment, Glial fibrillary acidic protein (GFAP) mediated expression or upregulation of a gene under the control of the GFAP promoter is by way of laser targeting. Other methods include activation using Cystatin C, a laser beam, radiation, transpupillary thermography or any other form of light energy in an amount sufficient to upregulate GFAP promoter expression in the GFAP-expressing cell type. Light, or photons are at a wavelength of from about 10 nm up to 2000 nm. Usually the wavelength is between about 500 nm to about 800 nm.

In another embodiment activation or upregulation of the GFAP promoter that drives the expression of the nucleic acid is via intravitreal or retrobulbar injections of factors that activate GFAP promoter, such as Cystatin C or elements of it that activate GFAP promoter, or other activators yet to be discovered.

In another preferred embodiment, the “laser-activatable domains of the GFAP promoter” are spliced into other suitable promoters, such as different viral promoters with varying strengths of activity may be utilized depending on the level of expression desired. Examples include: the CMV immediate early promoter if often used to provide strong transcriptional activation., modified versions of the CMV promoter that are less potent have also been used when reduced levels of expression of the transgene are desired, SV40, RSV LTR, HIV-1 and HIV-2 LTR, adenovirus promoters such as from the E1A, E2A, or MLP region, AAV LTR, cauliflower mosaic Virus, HSV-TK, and avian sarcoma virus.

Laser photocoagulation is an established treatment for a number of retinal neovascular diseases; however, the mechanism of its action is not yet fully understood. The same laser used in the treatment of retinal neovascular diseases is also used for the activation of the GFAP.

Vectors Including a GFAP Promoter

Another embodiment of the invention is a vector comprising the above-described purified nucleic acid construct including at least one nucleic acid encoding an anti-angiogenic protein, operably linked to a GFAP promoter. In embodiments of the vector designed for use in gene therapy, the vector can be an expression vector, such as a plasmid, or preferably a suitable viral vector such as an adenovirus vector, a recombinant adeno-associated virus (rAAV) vector, or a lentiviral vector. Adenovirus, while known as an effective vector, can elicit inflammatory responses and toxicity (Cartmell, T. et al.1999 J Neurosci 19:1517-23; Marshall, E. 2000 Science 288:955-7; Berry, M. et al. 2001 Curr Opin Mol Ther 3:338-49). By contrast, rAAV avoids these issues and therefore may be preferable.

With regard to ocular applications involving specific targeting of Muller cells of the retina with the rAAV vectors of the invention, it is known that two receptors necessary for rAAV to enter a cell are expressed on Muller cells, and that robust gene expression is observable in these cells as early as 16 hours after injection of rAAV vectors (Liang, F.-Q., et al in Retinal Degenerations: Mechansims and Experimental Therapy, M. LaVail, editor 2003, Kluwer Academic/Plenum Publishers, pp. 439-445) and can continue for at least 6 months (Kaplitt, M. G. et al. 1996 Ann Thorac Surg. 62:1669-76). Transfection of Muller cells has been previously achieved by injection of rAAV vectors into the vitreous. Using a glial cell-specific promoter (i.e., GFAP), the disclosed vectors provide the advantage of regulation of transgene expression in the cell type of interest, i.e., a glial cell such as a Muller cell in the retina. The results, shown in the Examples which follow, GFAP is rapidly upregulated in Muller cells in early stages of diabetic retinopathy, and in hypersensitive glaucoma.

Expression Vectors Including Hypoxia-Regulated Elements (HRE)

Some embodiments of the vectors include a hypoxia regulated element (HRE). Use of a HRE is highly advantageous for selective expression of a transgene in cells and tissues under conditions of hypoxia. Accordingly, expression of the transgene can be controlled by the physiological state of the transduced cell or tissue. More particularly, the gene therapy is selectively delivered to hypoxic foci in tissues, which foci are predisposed to develop neovascularization. The vectors of the invention, for example recombinant adeno-associated virus (rAAV) vectors containing HRE elements, can be constructed using methods proven successful for hypoxia-induced expression in cardiac myocytes. Some versions of the promoters further incorporate a HRE-silencer component (Prentice H. et al., Cardiovasc. Res. 35:567-574, 1997; Webster, K. A., Kubasiak, L. A., Prentice, H. and Bishopric, N. H.: Stable germline transmission of a hypoxia-activated molecular gene switch. From the double helix to molecular medicine, (ed. W. J. Whelan et al.), Oxford University Press, (2003). The HRE in the vectors activates the GFAP promoter to drive expression of the transgene in cells that express GFAP in response to hypoxia.

The nucleic acid constructs are readily incorporated into vectors suitable for gene therapy. Preferred embodiments of the vectors are recombinant adeno-associated virus (rAAV) vectors. Following construction, rAAV vectors are amplified in suitable cell lines, such as 293 cells, using techniques well known to those of skill in the art and further described in examples below. In some embodiments optimized for efficient transduction of retinal cells and rapid onset of expression, the strain rAAV 2/1 is preferred. Following transfection of the gene therapy vectors into GFAP-expressing cells, the vectors retain their cell-specific properties, normoxic silencing and hypoxia inducible characteristics (several hundred fold inducibility) in cell culture and in vivo.

GFAP-Driven Expression of Anti-Angiogenic Factors

The nucleic acid constructs and vectors of the invention include a GFAP promoter operably linked to a nucleic acid encoding an anti-angiogenic protein. Any one of a wide variety of proteins known to have anti-angiogenic properties, or similarly acting protein fragments or peptides derived from the sequences of these proteins can be used in the vectors (see, for example, Kuo C J et al., Proc. Natl. Acad. Sci. USA 98:4605-4610, 2001). In preferred embodiments of the vectors, the anti-angiogenic factors driven by a GFAP promoter include endostatin, angiostatin and Tubedown-1 Angiostatin is a proteolytic fragment of plasminogen known to inhibit endothelial cell proliferation and tumor growth (Cao, Y. et al., J. Biol. Chem. 271:29461-7, 1996; O'Reilly, M. S., EXS 79:273-294, 1997). Endostatin is a fragment of Collagen XVIII which advantageously inhibits neovascularization without toxicity to normal vessels (O'Reilly, M. S., et al., Cell 88: 277-85, 1997) and can effectively reduce macular edema (Takahashi K et al., Faseb J. 17:896-898, 2003). Vector-expressed angiostatin and endostatin have been shown to inhibit experimental neovascularization in vivo (Raisler B J et al., Proc. Natl. Acad. Sci. USA 99:8909-8914, 2002; Kuo C J et al., Proc. Natl. Acad. Sci. USA 98:4605-4610, 2001; Auricchio A. et al., Mol. Ther. 2002, 6:490-494). Tubedown-1 is an acetyltransferase associated with blood vessel development (Gendron R. L. et al., Dev. Dyn. 218:300-315, 2000).

To avoid widespread expression of the anti-angiogenic factors where undesirable, for example in the brain, in tissues undergoing wound healing or in the reproductive tract wherein normal angiogenesis may be required, some of the vectors are designed as described above to incorporate a HRE, to promote localized, hypoxia-regulated expression of the transgene.

In embodiments of the method involving ocular applications, the gene therapy to combat neovascularization in various compartments of the eye such as the vitreous, retina and choroid is achieved by expressing or over-expressing an anti-angiogenic protein in retinal Muller cells or other GFAP-expressing cell types of the eye, transduced with the vectors of the invention.

GFAP-Driven Gene Therapy for Neovascularization

The purified nucleic acid constructs and vectors of the invention are useful for gene therapy designed to target and prevent the growth of new blood vessels. Accordingly, another aspect of the invention is a gene therapy method, i.e., a method of preventing, reducing or delaying neovascularization. The method encompasses the steps of: (a) providing a subject having or at risk of developing a condition involving neovascularization in a tissue; and (b) transducing at least one GFAP-expressing cell type in the tissue of the subject with an expression vector including at least one nucleic acid encoding an anti-angiogenic protein operably linked to a GFAP promoter. Expression of the anti-angiogenic protein by the transduced cell prevents, reduces or delays neovascularization in the tissue of the subject.

The vectors and gene therapy methods of the invention can be used to treat neovascularization in any tissue of a subject's body containing cells that express GFAP. GFAP-expressing cells are typically distributed throughout the nervous system, including the retina of the eye. Nevertheless, the invention is suited to use in any GFAP-expressing cell, even those created recombinantly. Although examples herein describe ocular applications of the invention, the inventors do not intend to be so limited.

In a preferred embodiment, retinal disease treatment is directed to proliferative diabetic retinopathy (PDR) and/or glaucoma. However, retinal degenerations amenable to treatment disclosed in this invention include retinal degenerative diseases resulting in injury or death of retinal neurons such as photoreceptors and retinal ganglion cells. The retinal degenerative diseases include inherited, acquired, and inflammation induced retinal degenerative diseases. Inherited retinal degenerative diseases include, for example, all forms of macular degeneration such as dry and exudative age-related macular degeneration, Stargardt's disease, Best's disease, glaucoma, retinitis pigmentosa, and optic nerve degeneration. Acquired retinal degenerative diseases, for example, are caused by cystoid macular edema, retinal detachment, photic damage, ischemic retinopathies due to venous or arterial occlusion or other vascular disorders, retinopathies due to trauma, surgery, or penetrating lesions of the eye, and peripheral vitreoretinopathy. Inflammation-induced retinal degenerative diseases are caused by, for example, viral-, bacterial- and toxin-induced retinal degeneration, or uveitis, and may result in optic neuritis. This invention also provides a method for protecting against PDR and other retinal degenerations. The method can also be used in conjunction with other therapeutic modalities for treating retinal degenerations, including but not limited to administration with growth factors, neurotrophins, cytokines, ribozymes, anti-inflammatory agents, antibiotics, anti-viral agents, and gene therapy.

The invention is generally applicable to treatment of neovascularization arising from any cause, and is specifically contemplated to be useful for treatment of several ocular disorders of widely divergent etiology, including proliferative diabetic retinopathy (PDR) and choroidal neovascularization (CNV). Type I diabetes is associated with a high risk for PDR (Jacobsen, N. et al. 2003 Ugeskr Laeger 165:2953-6). Chronic exposure to the diabetic mileau typically leads to pre-proliferative retinopathy of several years' duration, characterized by extensive loss of retinal capillaries and cotton wool spots, followed by development of new vessels that grow from the retina into the normally avascular vitreous. The fragile new vessels are prone to leakage, causing macular edema and blurry vision. Susceptible to breakage, these abnormal vessels can result in immediate vision loss upon rupture. If permitted to grow, the neovascularization can form blinding fibrovascular membranes and cause the retina to detach. Under presently available treatment protocols, PDR is treated at the proliferative stage of the condition by placing a grid of laser burns over the retina. After a 20 year duration of diabetes, 33% of young adults have received such laser treatments, with associated decrease in visual acuity and visual angle (Kokkonen, J. et al. 1994 Acta Paediatr 83:273-8; Early Treatment Diabetic Retinopathy Study Research Group1991 Ophthalmology 98:766-85; Davies, N. 1999 Eye 13 (Pt 4):531-6; Dosso, A.A. et al. 2000 Diabetes Care 23:1855).

Pre-proliferative retinopathy is associated with focal areas of ischemia. It is widely accepted that neovascularization is associated with increased expression of pro-angiogenic factors such as vascular endothelial growth factor (VEGF), along with reduced expression of anti-angiogenic factors, such as endostatin and pigment epithelial derived factor, PEDF (Funatsu, H., et al. 2003 Invest Ophthalmol Vis Sci 44:1042-7; Noma, H., et al. 2002 Arch Ophthalmol 120:1075-80; Dawson, D. W. et al. 1999 Science 285:245-8; Spranger, J., et al. 2001 Diabetes 50:2641-5; Holekamp, N. M. et al 2002 Am J Ophthalmol 134:220-7; Boehm, B. O., et al. 2003 Horm Metab Res 35:382-6). The change in the balance between pro-angiogenic and anti-angiogenic factors elicits neovascularization and induces capillary leakage (Funatsu, H., et al. 2002 Am J Ophthalmol 133:70-7; Caldwell, R. B., et al. 2003 Diabetes Metab Res Rev19:442-55; Antcliff, R. J. et al. 1999 Semin Ophthalmol 14:223-32). Accordingly, it is specifically envisioned that the progression from pre-proliferative retinopathy to outright PDR can be significantly slowed by cell-specific, hypoxia-regulated expression of anti-angiogenic factors using the vectors and gene therapy methods of the invention.

Another condition that can be treated with the disclosed vectors and gene therapy methods is choroidal neovascularization (CNV). CNV is responsible for significant loss of vision, for example in age-related macular degeneration (AMD). In contrast to the situation in PDR, in the case of CNV abnormal new vessels grow from the choroid into the subretinal space. Retinas at high risk for CNV are identified by the presence of multiple or large soft drusen, reticular drusen, and/or pigmentary changes (Macular Photocoagulation Study Group 1997, Arch Ophthalmol 115:741-7; Arnold, J. J. et al. 1995 Retina 15:183-91). VEGF, ahypoxia-regulated protein, is associated with CNV (Frank, R. N., et al. 1996 Am J Ophthalmol 122:393-403; Ishibashi, T. et al. 1997 Graefes Arch Clin Exp Ophthalmol 235:159-67; Kwak, N. et al. 2000 Invest Ophthalmol Vis Sci 41:3158-64). Accordingly, it is likely that the neovascular lesions formed in CNV will be responsive to the hypoxia-regulated gene therapeutic compositions and approaches described herein.

Laser-Activated Gene Therapy for Neovascularization

Some embodiments of the method of gene therapy include an additional step (c) of irradiating the selected tissue with a laser beam sufficient to upregulate GFAP promoter expression in transduced GFAP-expressing cell types within the localized region affected by a laser treatment. It is known that following laser treatment, GFAP expression is elevated (Tassignon, M. J., et al. 1991 Graefes Arch Clin Exp Ophthalmol 229:380-8). The laser-activated embodiment of the method combines transduction of the tissue of interest with an anti-angiogenic vector of the invention, with subsequent selective upregulation of the GFAP promoter by laser irradiation. In this way, the level of expression of the introduced anti-angiogenic protein driven by the GFAP promoter can be increased, with both temporal and spatial control of the upregulation. The upregulation of the GFAP promoter by the laser is therefore, tissue specific and confined to the tissue which has been transduced by the vector. The localized increase in transgene expression is sufficient to prevent, reduce or delay neovascularization in the irradiated tissue. In ocular applications of the laser-activated version of the method, the irradiated tissue is within an eye. As is well known to those of skill in the art of ophthalmology, treatment of a variety of conditions with therapeutic laser beams is extensively practiced. Use of laser-activated gene therapy as described herein to permit precise control of the retinal region to be treated may be used either alone, or preferably as an adjuvant to regularly scheduled laser treatments for control of existing neovascular membranes. The result is a combined therapy that not only destroys existing vessels but activates the release of anti-angiogenic agents that can prevent the formation of new ones. Other methods include activation using Cystatin C, a laser beam, radiation, transpupillary thermography or any other form of light energy in an amount sufficient to upregulate GFAP promoter expression in the GFAP-expressing cell type. As used herein, “an amount sufficient” to upregulate, activate GFAP driven expression of a gene refers to light, or photons have a wavelength of from about 10 nm up to 2000 run. Usually the wavelength is between about 500 nm to about 800 nm.

In another embodiment, promoters that are activated by laser application include heat shock protein promoters. In this embodiment, a heat shock protein promoter can be used instead of the GFAP promoter.

Gene Therapy Vectors

As discussed above, a “vector” is any means for the transfer of a nucleic acid according to the invention into a host cell. Preferred vectors are viral vectors, such as adenoviruses and adeno-associated viruses, retroviruses, herpes viruses, plasmids, nucleic acids encapsulated in liposomes, RNA based vectors. Thus, a gene or nucleic acid sequence encoding an anti-angiogenic protein or polypeptide domain fragment thereof is introduced in vivo, ex vivo, or in vitro using a viral vector or through direct introduction of DNA, RNA. Expression in targeted tissues can be effected by targeting the transgenic vector to specific cells, such as with a viral vector or a receptor ligand, or by using a tissue-specific promoter, or both.

Viral vectors commonly used for in vivo or ex vivo targeting and therapy procedures are DNA-based vectors and viral vectors. A preferred vector is described in detail in the Examples which follow. Methods for constructing and using viral vectors are known in the art [see, e.g., Miller and Rosman, BioTechniques 7:980-990 (1992)]. Preferably, the viral vectors are replication defective, that is, they are unable to replicate autonomously in the target cell. In general, the genome of the replication defective viral vectors which are used within the scope of the present invention lack at least one region which is necessary for the replication of the virus in the infected cell. These regions can either be eliminated (in whole or in part), be rendered non-functional by any technique known to a person skilled in the art. These techniques include the total removal, substitution (by other sequences, in particular by the inserted nucleic acid), partial deletion or addition of one or more bases to an essential (for replication) region. Such techniques may be performed in vitro (on the isolated DNA) or in situ, using the techniques of genetic manipulation or by treatment with mutagenic agents. Preferably, the replication defective virus retains the sequences of its genome which are necessary for encapsulating the viral particles.

DNA viral vectors include an attenuated or defective DNA virus, such as but not limited to adenovirus, adeno-associated virus (AAV), herpes simplex virus (HSV), papillomavirus, Epstein-Barr virus (EBV), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV1) vector [Kaplitt et al., Molec. Cell. Neurosci. 2:320-330 (1991)], defective herpes virus vector lacking a glyco-protein L gene [Patent Publication RD 371005 A], or other defective herpes virus vectors [International Patent Publication No. WO 94/21807, published Sep. 29, 1994; International Patent Publication No. WO 92/05263, published Apr. 2, 1994]; an attenuated adenovirus vector, such as the vector described by Stratford-Perricaudet et al. [J. Clin. Invest. 90:626-630 (1992); see also La Salle et al., Science 259:988-990 (1993)]; and a defective adeno-associated virus vector [Samulski et al., J. Virol. 61:3096-3101 (1987); Samulski et al., J. Virol. 63:3822-3828 (1989); Lebkowski et al., Mol. Cell. Biol. 8:3988-3996 (1988)].

Preferably, for in vivo administration, an appropriate immunosuppressive treatment is employed in conjunction with the viral vector, e.g., adenovirus vector, to avoid immuno-deactivation of the viral vector and transfected cells. For example, immunosuppressive cytokines, such as interleukin-12 (IL-12), interferon-y (IFN-γ), or anti-CD4 antibody, can be administered to block humoral or cellular immune responses to the viral vectors [see, e.g., Wilson, Nature Medicine (1995)]. In addition, it is advantageous to employ a viral vector that is engineered to express a minimal number of antigens.

Adenovirus vectors: in one preferred embodiment, the vector is an adenovirus vector. Defective adenovirus vectors are effective for delivery of the angiogenesis inhibitors, for example ATF and angiostatin. Adenoviruses are eukaryotic DNA viruses that can be modified to efficiently deliver a nucleic acid of the invention to a variety of cell types. Various serotypes of adenovirus exist. Of these serotypes, preference is given, within the scope of the present invention, to using type 2 or type 5 human adenoviruses (Ad 2 or Ad 5) or adenoviruses of animal origin (see WO94/26914). Those adenoviruses of animal origin which can be used within the scope of the present invention include adenoviruses of canine, bovine, murine (example: Mavl, Beard 75 (1990) 81), ovine, porcine, avian, and simian (example: SAV) origin. The adenovirus of animal origin is a canine adenovirus, more preferably a CAV2 adenovirus (e.g., Manhattan or A26/61 strain (ATCC VR-800), for example).

Preferably, the replication defective adenoviral vectors of the invention comprise the ITRs, an encapsidation sequence and the nucleic acid of interest. Still more preferably, at least the E1 region of the adenoviral vector is non-functional. The deletion in the E1 region preferably extends from nucleotides 455 to 3329 in the sequence of the Ad5 adenovirus (PvuII-BgIII fragment) or 382 to 3446 (HinfII-Sau3A fragment). Other regions may also be modified, in particular the E3 region (WO95/02697), the E2 region (WO94/28938), the E4 region (WO94/28152, WO94/12649 and WO95/02697), or in any of the late genes L1-L5.

In a preferred embodiment, the adenoviral vector has a deletion in the E1 region (Ad 1.0). Examples of E1-deleted adenoviruses are disclosed in EP 185,573, the contents of which are incorporated herein by reference. In another preferred embodiment, the adenoviral vector has a deletion in the E1 and E4 regions (Ad 3.0). Examples of E1/E4-deleted adenoviruses are disclosed in WO95/02697 and WO96/22378, the contents of which are incorporated herein by reference. In still another preferred embodiment, the adenoviral vector has a deletion in the E1 region into which the E4 region and the nucleic acid sequence are inserted.

The replication defective recombinant adenoviruses according to the invention can be prepared by any technique known to the person skilled in the art (Levrero et al., Gene 101 (1991) 195, EP 185 573; Graham, EMBO J. 3 (1984) 2917). In particular, they can be prepared by homologous recombination between an adenovirus or modified adenovirus genome and a plasmid which carries, inter alia, the DNA sequence of interest. The homologous recombination is effected following cotransfection of the said adenovirus and plasmid into an appropriate cell line. The cell line which is employed should preferably (i) be transformable by the said elements, and (ii) contain the sequences which are able to complement the part of the genome of the replication defective adenovirus, preferably in integrated form in order to avoid the risks of recombination. Examples of cell lines which may be used are the human embryonic kidney cell line 293 (Graham et al., J. Gen. Virol. 36 (1977) 59) which contains the left-hand portion of the genome of an Ad5 adenovirus (12%) integrated into its genome, and cell lines which are able to complement the E1 and E4 functions, as described in applications WO94/26914 and WO95/02697. Recombinant adenoviruses are recovered and purified using standard molecular biological techniques, which are well known to one of ordinary skill in the art.

Adeno-associated viruses. In a preferred embodiment, the vector is an adeno-associated viruses. An example of an exemplary vector is described in detail in the Examples which follow. Briefly, hypoxia-regulated elements to physiologically regulate the expression of the transfected gene. Placing multiple copies of an hypoxia-responsive elements upstream of luciferase (a reporter gene) had only small effects on gene expression in room air, but resulted in 5 to 6 fold increase in luciferase expression when the transfected cultures were placed in hypoxia (8-12 mm Hg). Luciferase expression in transfected cardiac muscle increased 5-fold an hour after ligation of an artery, and remained similarly elevated for 4 hours after the heart was reperfused. Constructs lacking the hypoxia responsive element had low levels of constitutive expression, and exhibited no responsive to hypoxia.

Addition of HRE to rAAV -VEGF vectors supported even greater response to hypoxia. In responsive to ischemia, VEGF expression increased 16 fold in hearts transfected with rAAV-VEGF, and 199-fold in hearts transfected with rAAV-HRE(9)-VEGF (containing a concatamer of 9 HRE consensus sequences).

The adeno-associated viruses (AAV) are DNA viruses of relatively small size which can integrate, in a stable and site-specific manner, into the genome of the cells which they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions which carry the encapsidation functions: the left-hand part of the genome, which contains the rep gene involved in viral replication and expression of the viral genes; and the right-hand part of the genome, which contains the cap gene encoding the capsid proteins of the virus.

The use of vectors derived from the AAVs for transferring genes in vitro and in vivo has been described (see WO 91/18088; WO 93/09239; U.S. Pat. No. 4,797,368, U.S. Pat. No. 5,139,941, EP 488 528). These publications describe various AAV-derived constructs in which the rep and/or cap genes are deleted and replaced by a gene of interest, and the use of these constructs for transferring the said gene of interest in vitro (into cultured cells) or in vivo, (directly into an organism). The replication defective recombinant AAVs according to the invention can be prepared by cotransfecting a plasmid containing the nucleic acid sequence of interest flanked by two AAV inverted terminal repeat (ITR) regions, and a plasmid carrying the AAV encapsidation genes (rep and cap genes), into a cell line which is infected with a human helper virus (for example an adenovirus). The AAV recombinants which are produced are then purified by standard techniques.

The invention also relates, therefore, to an AAV-derived recombinant virus whose genome encompasses a sequence encoding a nucleic acid encoding an anti-angiogenic factor flanked by the AAV ITRs. The invention also relates to a plasmid encompassing a sequence encoding a nucleic acid encoding an anti-angiogenic factor flanked by two ITRs from an AAV. Such a plasmid can be used as it is for transferring the nucleic acid sequence, with the plasmid, where appropriate, being incorporated into a liposomal vector (pseudo-virus).

Retrovirus vectors: in another embodiment the gene can be introduced in a retroviral vector, e.g., as described in Anderson et al., U.S. Pat. No. 5,399,346; Mann et al., 1983, Cell 33:153; Temin et al., U.S. Pat. No. 4,650,764; Temin et al., U.S. Pat. No. 4,980,289; Markowitz et al., 1988, J. Virol. 62:1120; Temin et al., U.S. Pat. No. 5,124,263; EP 453242, EP178220; Bernstein et al. Genet. Eng. 7 (1985) 235; McCormick, BioTechnology 3 (1985) 689; International Patent Publication No. WO 95/07358, published Mar. 16, 1995, by Webster, K. A., Kubasiak, L. A., Prentice, H. and Bishopric, N. H.: Stable germline transmission of a hypoxia-activated molecular gene switch. From the double helix to molecular medicine, (ed. W. J. Whelan et al.), Oxford University Press, (2003); and Kuo et al., 1993, Blood 82:845. The retroviruses are integrating viruses which infect dividing cells. The retrovirus genome includes two LTRs, an encapsidation sequence and three coding regions (gag, pol and env). In recombinant retroviral vectors, the gag, pol and env genes are generally deleted, in whole or in part, and replaced with a heterologous nucleic acid sequence of interest. These vectors can be constructed from different types of retrovirus, such as, HIV, MoMuLV (“murine Moloney leukaemia virus” MSV (“murine Moloney sarcoma virus”), HaSV (“Harvey sarcoma virus”); SNV (“spleen necrosis virus”); RSV (“Rous sarcoma virus”) and Friend virus. Defective retroviral vectors are disclosed in WO95/02697.

In general, in order to construct recombinant retroviruses containing a nucleic acid sequence, a plasmid is constructed which contains the LTRs, the encapsidation sequence and the coding sequence. This construct is used to transfect a packaging cell line, which cell line is able to supply in trans the retroviral functions which are deficient in the plasmid. In general, the packaging cell lines are thus able to express the gag, pol and env genes. Such packaging cell lines have been described in the prior art, in particular the cell line PA317 (U.S. Pat. No. 4,861,719); the PsiCRIP cell line (WO90/02806) and the GP⁺ envAm-12 cell line (WO89/07150). In addition, the recombinant retroviral vectors can contain modifications within the LTRs for suppressing transcriptional activity as well as extensive encapsidation sequences which may include a part of the gag gene (Bender et al., J. Virol. 61 (1987) 1639). Recombinant retroviral vectors are purified by standard techniques known to those having ordinary skill in the art.

Retroviral vectors can be constructed to function as infectious particles or to undergo a single round of transfection. In the former case, the virus is modified to retain all of its genes except for those responsible for oncogenic transformation properties, and to express the heterologous gene. Non-infectious viral vectors are prepared to destroy the viral packaging signal, but retain the structural genes required to package the co-introduced virus engineered to contain the heterologous gene and the packaging signals. Thus, the viral particles that are produced are not capable of producing additional virus.

Targeted gene delivery is described in International Patent Publication WO 95/28494, published October 1995.

Lentiviral Vectors: lentiviruses include members of the bovine lentivirus group, equine lentivirus group, feline lentivirus group, ovinecaprine lentivirus group and primate lentivirus group. The development of lentiviral vectors for gene therapy has been reviewed in Klimatcheva et al., 1999, Frontiers in Bioscience 4: 481-496. The design and use of lentiviral vectors suitable for gene therapy is described, for example, in U.S. Pat. No. 6,207,455, issued Mar. 27, 2001, and U.S. Pat. No. 6,165,782, issued Dec. 26, 2000. Examples of lentiviruses include, but are not limited to, HIV-1, HIV-2, HIV-1/HIV-2 pseudotype, HIV-1/SIV, FIV, caprine arthritis encephalitis virus (CAEV), equine infectious anemia virus and bovine immunodeficiency virus. HIV-1 is preferred.

Non-viral Vectors: alternatively, the vector can be introduced in vivo as nucleic acid free of transfecting excipients, or with transfection facilitating agents, e.g., lipofection. For the past decade, there has been increasing use of liposomes for encapsulation and transfection of nucleic acids in vitro. Synthetic cationic lipids designed to limit the difficulties and dangers encountered with liposome mediated transfection can be used to prepare liposomes for in vivo transfection of a gene encoding a marker [Feigner, et. al., Proc. Natl. Acad. Sci. U.S.A. 84:7413-7417 (1987); see Mackey, et al., Proc. Natl Acad. Sci. U.S.A. 85:8027-8031 (1988); Ulmer et al., Science 259:1745-1748 (1993)]. The use of cationic lipids may promote encapsulation of negatively charged nucleic acids, and also promote fusion with negatively charged cell membranes [Feigner and Ringold, Science 337:387-388 (1989)]. Particularly useful lipid compounds and compositions for transfer of nucleic acids are described in International Patent Publications WO95/18863 and WO96/17823, and in U.S. Pat. No. 5,459,127. The use of lipofection to introduce exogenous genes into the specific organs in vivo has certain practical advantages. Molecular targeting of liposomes to specific cells represents one area of benefit. It is clear that directing transfection to particular cell types would be particularly advantageous in a tissue with cellular heterogeneity, such as pancreas, liver, kidney, and the brain. Lipids may be chemically coupled to other molecules for the purpose of targeting [see Mackey, et. al., supra]. Targeted peptides, e.g., hormones or neurotransmitters, and proteins such as antibodies, or non-peptide molecules could be coupled to liposomes chemically.

Other molecules are also useful for facilitating transfection of a nucleic acid in vivo, such as a cationic oligopeptide (e.g., International Patent Publication WO95/2193 1), peptides derived from DNA binding proteins (e.g., International Patent Publication WO96/25508), or a cationic polymer (e.g., International Patent Publication WO95/21931).

It is also possible to introduce the vector in vivo as a naked DNA plasmid. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun, or use of a DNA vector transporter [see, e.g., Wu et al., J. Biol. Chem. 267:963-967 (1992); Wu and Wu, J. Biol. Chem. 263:14621-14624 (1988); Williams et al., Proc. Natl. Acad. Sci. USA 88:2726-2730 (1991)]. Receptor-mediated DNA delivery approaches can also be used [Curiel et al., Hum. Gene Ther. 3:147-154(1992); Wu and Wu, J Biol. Chem. 262:4429-4432 (1987)].

The nucleic acid can also be administered as a naked DNA. Methods for formulating and administering naked DNA to mammalian muscle tissue are disclosed in U.S. Pat. Nos. 5,580,859 and 5,589,466, the contents of which are incorporated herein by reference.

Regulatory Regions: expression of an anti-angiogenic factor from a vector of the invention may be controlled by any regulatory region, i.e., promoter/enhancer element known in the art. The regulatory regions may comprise a promoter region for ftnctional transcription in the retina, as well as a region situated in 3′ of the gene of interest, and which specifies a signal for termination of transcription and a polyadenylation site. All these elements constitute an expression cassette.

Promoters that may be used in the present invention, besides the GFAP include both constitutive promoters and regulated (inducible) promoters. The promoter may be naturally responsible for the expression of the nucleic acid. It may also be from a heterologous source. In particular, it may be promoter sequences of eukaryotic or viral genes. For example, it may be promoter sequences derived from the genome of the cell which it is desired to infect. Likewise, it may be promoter sequences derived from the genome of a virus, including the adenovirus used. In this regard, there may be mentioned, for example, the promoters of the E1A, MLP, CMV and RSV genes and the like.

In addition, the promoter may be modified by addition of activating or regulatory sequences or sequences allowing a tissue-specific or predominant expression (enolase and GFAP promoters and the like). Moreover, when the nucleic acid does not contain promoter sequences, it may be inserted, such as into the virus genome downstream of such a sequence. Some promoters useful for practice of this invention are heat shock protein promoters (hsp), ubiquitous promoters (e.g., HPRT, vimentin, actin, tubulin), intermediate filament promoters (e.g., GFAP, desmin, neurofilaments, keratin), therapeutic gene promoters (e.g., MDR type, CFTR, factor VIII), tissue-specific promoters (e.g., actin promoter in smooth muscle cells), promoters which are preferentially activated in dividing cells, promoters which respond to a stimulus (e.g., steroid hormone receptor, retinoic acid receptor), tetracycline-regulated transcriptional modulators, cytomegalovirus immediate-early, retroviral LTR, metallothionein, SV-40, E1A, and MLP promoters. Tetracycline-regulated transcriptional modulators and CMV promoters are described in WO 96/01313, U.S. Pat. Nos. 5,168,062 and 5,385,839, the contents of which are incorporated herein by reference.

Thus, the promoters which may be used to control gene expression include, but are not limited to, GFAP, HSP promoters, the cytomegalovirus (CMV) promoter, the SV40 early promoter region (Benoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42); prokaryotic expression vectors such as the β-lactamase promoter (Villa-Kamaroff, et al., 1978, Proc. Natl. Acad. Sci. U.S.A. 75:3727-3731), or the tac promoter (DeBoer, et al., 1983, Proc. Natl. Acad. Sci. U.S.A. 80:21-25); promoter elements from yeast or other fungi such as the Gal 4 promoter, the ADC (alcohol dehydrogenase) promoter, PGK (phosphoglycerol kinase) promoter, alkaline phosphatase promoter.

Vectors of the invention can be administered to a subject typically by injection into the vitreous or subretinal space using a fine needle. The amount of vector to be delivered depends on the size of the eye, how extensive an area was being targeted and is readily determined by a practioner. When administered it is preferred that the vectors be given in a pharmaceutical vehicle suitable for injection such as a sterile aqueous solution or dispersion. Following administration, the subject is monitored to detect changes in gene expression. Dose and duration of treatment is determined individually depending on the condition or disease to be treated. A wide variety of conditions or diseases can be treated based on the gene expression produced by administration of the gene of interest in the vector of the present invention. The dosage of vector delivered using the method of the invention will vary depending on the desired response by the host and the vector used. Generally, it is expected that up to 100-200 μg of DNA or RNA can be administered in a single dosage, although a range of 0.5 mg/kg body weight to 50 mg/kg body weight will be suitable for most applications.

Enhancers

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

In preferred embodiments, the promoters can comprise enhancers.

Below is a list of promoters additional to the tissue specific promoters listed above, cellular promoters/enhancers and inducible promoters/enhancers that could be; used in combination with the nucleic acid encoding a gene of interest in an expression construct. Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 1
ENHANCER

Immunoglobulin Heavy Chain
Immunoglobulin Light Chain
T-Cell Receptor
HLA DQα and DQβ
β-Interferon
Interleukin-2
Interleukin-2 Receptor
MHC Class II HLA-DRα
β-Actin
Muscle Creatine Kinase
Prealbumin (Transthyretin)
Elastase I
Metallothionein
Collagenase
Albumin Gene
α-Fetoprotein
τ-Globin
β-Globin
e-fos
c-HA-ras
Insulin
Neural Cell Adhesion Molecule (NCAM)
α1-Antitrypsin
H2B (TH2B) Histone
Mouse or Type I Collagen
Glucose-Regulated Proteins (GRP94 and GRP78)
Rat Growth Hormone
Human Serum Amyloid A (SAA)
Troponin I (TN I)
Platelet-Derived Growth Factor
Duchenne Muscular Dystrophy
SV40
Polyoma
Retroviruses
Papilloma Virus
Hepatitis B Virus
Human Immunodeficiency Virus
Cytomegalovirus
Gibbon Ape Leukemia Virus

TABLE 2

| Element | Inducer |
|---|---|
| MT II | Phorbol Ester (TPA) |
| | Heavy metals |
| MMTV (mouse mammary tumor | Glucocorticoids |
| virus) | |
| β-Interferon | poly(rI)X |
| | poly(rc) |
| Adenovirus 5 E2 | E1A |
| c-jun | Phorbol Ester (TPA), H₂O₂ |
| Collagenase | Phorbol Ester (TPA) |
| Stromelysin | Phorbol Ester (TPA), IL-1 |
| SV40 | Phorbol Ester (TPA) |
| Murine MX Gene | Interferon, Newcastle Disease Virus |
| GRP78 Gene | A23187 |
| α-2-Macroglobulin | IL-6 |
| Vimentin | Serum |
| MHC Class I Gene H-2kB | Interferon |
| HSP70 | E1A, SV40 Large T Antigen |
| Proliferin | Phorbol Ester-TPA |
| Tumor Necrosis Factor | FMA |
| Thyroid Stimulating Hormone | α-Thyroid Hormone |
| Gene | |
| Insulin E Box | Glucose |

In preferred embodiments of the invention, the expression construct comprises a virus or engineered construct derived from a viral genome. Viral genomes include herpes viruses into which large locus control regions are incorporated into the vectors, such that they function as master regulators.

“Transcriptional regulatory sequence” is a generic term used throughout the specification to refer to DNA sequences, such as initiation signals, enhancers, and promoters, as well as polyadenylation sites, which induce or control transcription of protein (or antisense) coding sequences with which they are operably linked. In preferred embodiments, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of the regulatory protein.

The heterologous regulatory sequences, e.g., which are provided in the replacement region, can include one or more of a variety elements, including: promoters (such as constitutive or inducible promoters), enhancers, negative regulatory elements, locus control regions, transcription factor binding sites, or combinations thereof. Promoters/enhancers which may be used to control the expression of the targeted gene in vivo include, but are not limited to, the cytomegalovirus (CMV) promoter/enhancer (Karasuyama et al., 1989, J. Exp. Med., 169:13), the human β-actin promoter (Gunning et al. (1987) PNAS 84:4831-4835), the glucocorticoid-inducible promoter present in the mouse mammary tumor virus long terminal repeat (MMTV LTR) (Klessig et al. (1984) Mol. Cell Biol. 4:1354-1362), the long terminal repeat sequences of Moloney murine leukemia virus (MuLV LTR) (Weiss et al. (1985) RNA Tumor Viruses, Cold Spring Harbor Laboratory, Cold Spring Harbor, N.Y.), the SV40 early or late region promoter (Bernoist et al. (1981) Nature 290:304-310; Templeton et al. (1984) Mol. Cell Biol., 4:817; and Sprague et al. (1983) J. Virol., 45:773), the promoter contained in the 3′ long terminal repeat of Rous sarcoma virus (RSV) (Yamanoto et al., 1980, Cell, 22:787-797), the herpes simplex virus (HSV) thymidine kinase promoter/enhancer (Wagner et al. (1981) PNAS 82:3567-71), and the herpes simplex virus LAT promoter (Wolfe et al. (1992) Nature Genetics, 1:379-384).

Techniques for the formation of vectors or virions, for delivering a gene of interest to a target cell of interest are generally described in “Working Toward Human Gene Therapy,” Chapter 28 in Recombinant DNA, 2nd Ed., Watson, J. D. et al., eds., New York: Scientific American Books, pp. 567-581 (1992). An overview of viral vectors or virions that have been used in gene therapy can be found in Wilson, J. M., Clin. Exp. Immunol. 107(Suppl. 1):31-32 (1997), as well as Nakanishi, M, Crit. Rev. Therapeu. Drug Carrier Systems 12:263-310 (1995); Robbins, P. D., et al., Trends Biotechnol. 16:35-40 (1998); Zhang, J., et al., Cancer Metastasis Rev. 15:385-401 (1996); and Kramm, C. M., et al., Brain Pathology 5:345-381 (1995). Such vectors may be derived from viruses that contain RNA (Vile, R. G., et al., Br. Med Bull. 51:12-30 (1995)) or DNA (Ali M., et al., Gene Ther. 1:367-384 (1994)) and HSV vectors described in U.S. Pat. No. 6,573,090, incorporated herein by reference.

Specific examples of viral vector systems that have been utilized in the gene therapy art include: retroviruses (Vile, R. G., supra; U.S. Pat. Nos. 5,741,486 and 5,763,242); adenoviruses (Brody, S. L., et al., Ann. N.Y. Acad. Sci. 716: 90-101 (1994); Heise, C. et al., Nat. Med. 3:639-645 (1997)); adenoviral/retroviral chimeras (Bilbao, G., et al., FASEB J. 11:624-634 (1997); Feng, M., et al., Nat. Biotechnol. 15:866-870 (1997)); adeno-associated viruses (Flotte, T. R. and Carter, B. J., Gene Ther. 2:357-362 (1995); U.S. Pat. No. 5,756,283); herpes simplex virus I or II (Latchman, D. S., Mol. Biotechnol. 2:179-195 (1994); U.S. Pat. No. 5,763,217; Chase, M., et al., Nature Biotechnol. 16:444-448 (1998)); parvovirus (Shaughnessy, E., et al., Semin Oncol. 23:159-171 (1996)); reticuloendotheliosis virus (Donburg, R., Gene Therap. 2:301-310 (1995)). Other viruses that can be used as vectors for gene delivery include poliovirus, papillomavirus, vaccinia virus, lentivirus, as well as hybrid or chimeric vectors incorporating favorable aspects of two or more viruses (Nakanishi, M, Crit. Rev. Therapeu. Drug Carrier Systems 12:263-310 (1995); Zhang, J., et al., Cancer Metastasis Rev. 15:385-401 (1996); Jacoby, D. R., et al., Gene Therapy 4:1281-1283 (1997)). Guidance in the construction of gene therapy vectors and the introduction thereof into affected animals for therapeutic purposes may be obtained in the above-referenced publications, as well as U.S. Pat. Nos. 6,573,090, 5,631,236, 5,688,773, 5,691,177, 5,670,488, 5,529,774, 5,601,818, and WO 95/06486.

Polyadenylation Signals

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human or bovine growth hormone and SV40 polyadenylation signals. Also contemplated as an element of the expression cassette is a terminator. These elements can serve to enhance message levels and to minimize read through from the cassette into other sequences.

IRES

In certain embodiments of the invention, the use of internal ribosome entry site (IRES) elements is contemplated to create multigene, or polycistronic, messages. IRES elements are able to bypass the ribosome scanning model of 5′ methylated Cap dependent translation and begin translation at internal sites. IRES elements from two members of the picomavirus family (poliovirus and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

Assessing Gene Silencing

Transfer of an exogenous nucleic acid into a host cell or organism by a vector can be assessed by directly detecting the presence of the nucleic acid in the cell or organism. Such detection can be achieved by several methods well known in the art. For example, the presence of the exogenous nucleic acid can be detected by Southern blot or by a polymerase chain reaction (PCR) technique using primers that specifically amplify nucleotide sequences associated with the nucleic acid. Expression of the exogenous nucleic acids can also be measured using conventional methods. For instance, mRNA produced from an exogenous nucleic acid can be detected and quantified using a Northern blot and reverse transcription PCR (RT-PCR).

Expression of an RNA from the exogenous nucleic acid can also be detected by measuring an enzymatic activity or a reporter protein activity. For example, siRNA activity can be measured indirectly as a decrease in target nucleic acid expression as an indication that the exogenous nucleic acid is producing the effector RNA.

Genes Encoding Anti-Angiogenic Proteins

The vectors of the invention can be used to deliver a gene encoding an anti-angiogenic protein into the retina in accordance with the invention. In a preferred embodiment, the anti-angiogenic factors are endostatin, angiostatin, Tubedown-1 and the amino terminal fragment (ATF) of urokinase, containing the EGF-like domain. Such fragment corresponds to amino acid residues about 1 to about 135 of ATF.

Genes encoding other anti-angiogenesis protein can also be used according to the invention. Such genes include, but are not limited to, genes encoding angiostatin [O'Reilly et al., Cell 79:315-328 (1994); WO95/29242; U.S. Pat. No. 5,639,725], including angiostatin comprising kringles 1 to 3; tissue inhibition of metalloproteinase [Johnson et al., J. Cell. Physiol. 160:194-202 (1994)]; inhibitors of FGF or VEGF; and endostatin [WO97/15666], stroam1 derived factor 1 (SDF-1) and 16 kDa N-terminal fragments of human prolactin. In another preferred embodiment, the amino terminal fragment of plasminogen/angiostatin is human plasminogen (angiostatin).

In another embodiment, the invention provides for administration of genes encoding soluble forms of receptors for angiogenic factors, including but not limited to soluble VGF/VEGF receptor, and soluble urokinase receptor [Wilhem et al., FEBS Letters 337:131-134 (1994)]. In general, any gene encoding a protein or soluble receptor that antagonizes endothelial cell activation and migration, which is involved in angiogenesis, can be employed in the gene therapy vectors and methods of the invention. Notwithstanding, it is particularly noteworthy that gene therapy delivery of endostatin, angiostatin and Tubedown-1 is especially effective in this regard, for reasons pointed out above and exemplified below.

A gene encoding an anti-angiogenic factor, whether genomic DNA or cDNA, can be isolated from any source, particularly from a human cDNA or genomic library. Methods for obtaining such genes are well known in the art, as described above [see, e.g., Sambrook et al., 1989, supra]. Due to the degeneracy of nucleotide coding sequences, other nucleic acid sequences which encode substantially the same amino acid sequence as an anti-angiogenic factor gene may be used in the practice of the present invention and these are contemplated as falling within the scope of the claimed invention. These include but are not limited to allelic genes, homologous genes from other species, and nucleotide sequences comprising all or portions of anti-angiogenic factor genes which are altered by the substitution of different codons that encode the same amino acid residue within the sequence, thus producing a silent change. Likewise, the anti-angiogenic factor derivatives of the invention include, but are not limited to, those containing, as a primary amino acid sequence, all or part of the amino acid sequence of an anti-angiogenic factor protein including altered sequences in which functionally equivalent amino acid residues are substituted for residues within the sequence resulting in a conservative amino acid substitution. For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such alterations will not be expected to affect apparent molecular weight as determined by polyacrylamide gel electrophoresis, or isoelectric point.

Particularly preferred substitutions are: Lys for Arg and vice versa such that a positive charge may be maintained; Glu for Asp and vice versa such that a negative charge may be maintained; Ser for Thr such that a free —OH can be maintained; and Gln for Asn such that a free CONH₂ can be maintained.

The genes encoding anti-angiogenic factor derivatives and analogs of the invention can be produced by various methods known in the art. The manipulations which result in their production can occur at the gene or protein level. For example, the cloned anti-angiogenic factor gene sequence can be modified by any of numerous strategies known in the art (Sambrook et al., 1989, supra). The sequence can be cleaved at appropriate sites with restriction endonuclease(s), followed by further enzymatic modification if desired, isolated, and ligated in vitro. In the production of the gene encoding a derivative or analog of anti-angiogenic factor, care should be taken to ensure that the modified gene remains within the same translational reading frame as the anti-angiogenic factor gene, uninterrupted by translational stop signals, in the gene region where the desired activity is encoded.

Additionally, the anti-angiogenic factor-encoding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification, such as to form a chimeric gene: Preferably, such mutations enhance the functional activity of the mutated anti-angiogenic factor gene product. Any technique for mutagenesis known in the art can be used, including but not limited to, in vitro site-directed mutagenesis (Hutchinson, C., et al., 1978, J. Biol. Chem. 253:6551; Zoller and Smith, 1984, DNA 3:479-488; Oliphant et al., 1986, Gene 44:177; Hutchinson et al., 1986, Proc. Natl. Acad. Sci. U.S.A. 83:710), use of TAB™ linkers (Pharmacia), etc. PCR techniques are preferred for site directed mutagenesis (see Higuchi, 1989, “Using PCR to Engineer DNA”, in PCR Technology: Principles and Applications for DNA Amplification, H. Erlich, ed., Stockton Press, Chapter 6, pp. 61-70).

Therapeutic Molecules

In preferred embodiments, the anti-angiogenic protein expressed by the vectors are angiostatin, endostatin or Tubedown-1.

However, a wide variety of molecules may be utilized within the scope of the present invention as anti-angiogenic factors, including for example Anti-Invasive Factor, retinoic acids and their derivatives, paclitaxel including analogues and derivatives thereof, Suramin, Tissue Inhibitor of Metalloproteinase-1, Tissue Inhibitor of Metalloproteinase-2, Plasminogen Activator Inhibitor-I and Plasminogen Activator Inhibitor-2, and lighter “d group” transition metals. Similarly, a wide variety of polymeric carriers may be utilized, representative examples of which include poly (ethylene-vinyl acetate) (40% cross-linked), poly (D,L-lactic acid) oligomers and polymers, poly (L-lactic acid) oligomers and polymers, poly (glycolic acid), copolymers of lactic acid and glycolic acid, poly (caprolactone), poly (valerolactone), poly (anhydrides), copolymers of poly (caprolactone) or poly (lactic acid) with polyethylene glycol, and blends thereof. As noted above, the present invention provides compositions comprising an anti-angiogenic factor, and a polymeric carrier. Briefly, a wide variety of anti-angiogenic factors may be readily utilized within the context of the present invention. Representative examples include Anti-Invasive Factor, retinoic acid and derivatives thereof, paclitaxel, Suramin, Tissue Inhibitor of Metalloproteinase-1, Tissue Inhibitor of Metalloproteinase-2, Plasminogen Activator Inhibitor-1, Plasminogen Activator Inhibitor-2, and various forms of the lighter “d group” transition metals. These and other anti-angiogenic factors will be discussed in more detail below.

Briefly, Anti-Invasive Factor, or “AIF” which is prepared from extracts of cartilage, contains constituents which are responsible for inhibiting the growth of new blood vessels. These constituents comprise a family of 7 low molecular weight proteins (<50,000 daltons) (Kuettner and Pauli, “Inhibition of neovascularization by a cartilage factor” in Development of the Vascular System, Pitman Books (CIBA Foundation Symposium 100), pp. 163-173, 1983), including a variety of proteins which have inhibitory effects against a variety of proteases (Eisentein et al, Am. J Pathol. 81:337-346, 1975; Langer et al., Science 193:70-72, 1976; and Horton et al., Science 199:1342-1345, 1978). AIF suitable for use within the present invention may be readily prepared utilizing techniques known in the art (e.g., Eisentein et al, supra; Kuettner and Pauli, supra; and Langer et al., supra). Purified constituents of AIF such as Cartilage-Derived Inhibitor (“CDI”) (see Moses et al., Science 248:1408-1410, 1990) may also be readily prepared and utilized within the context of the present invention.

Cartilage derived inhibitors include those described by Sorgente N, Dorey C. Inhibition of endothelial cell growth by a factor isolated from cartilage. Exp Cell Res. 1980;128:63-71, which is incorporated herein by reference in its entirety.

Combination therapy using the vectors of the invention is in accordance with the invention. The following can be used in conjunction with the vectors.

Retinoic acids alter the metabolism of extracellular matrix components, resulting in the inhibition of angiogenesis. Retinoic acid, as well as derivatives thereof which may also be utilized in the context of the present invention, may be readily obtained from commercial sources, including for example, Sigma Chemical Co. (# R2625).

Paclitaxel is a highly derivatized diterpenoid (Wani et al., J. Am. Chem. Soc. 93:2325, 1971) which has been obtained from the harvested and dried bark of Taxis brevifolia (Pacific Yew.) and Taxomyces Andreanae and Endophytic Fungus of the Pacific Yew (Stierle et al., Science 60:214-216, 1993). Generally, paclitaxel acts to stabilize microtubular structures by binding tubulin to form abnormal mitotic spindles. “Paclitaxel” (which should be understood herein to include analogues and derivatives such as, for example, TAXOL™, TAXOTERE™, 10-desacetyl analogues of paclitaxel and 3′N-desbenzoyl-3′N-t-butoxy carbonyl analogues of paclitaxel) may be readily prepared utilizing techniques known to those skilled in the art (see also WO 94/07882, WO 94/07881, WO 94/07880, WO 94/07876, WO 93/23555, WO 93/10076, U.S. Pat. Nos. 5,294,637, 5,283,253, 5,279,949, 5,274,137, 5,202,448, 5,200,534, 5,229,529, and EP 590267), or obtained from a variety of commercial sources, including for example, Sigma Chemical Co., St. Louis, Mo. (T7402-from Taxus brevifolia).

Suramin is a polysulfonated naphthylurea compound that is typically used as a trypanocidal agent. Briefly, Suramin blocks the specific cell surface binding of various growth factors such as platelet derived growth factor (“PDGF”), epidermal growth factor (“EGF”), transforming growth factor (“TGF-β”), insulin-like growth factor (“IGF-1”), and fibroblast growth factor (“βFGF”). Suramin may be prepared in accordance with known techniques, or readily obtained from a variety of commercial sources, including for example Mobay Chemical Co., New York. (see Gagliardi et al., Cancer Res. 52:5073-5075, 1992; and Coffey, Jr., et al., J. of Cell. Phys. 132:143-148, 1987).

Tissue inhibitor of metalloproteinases can be expressed in the expression vector and/or administered in conjunction with the compositions of the invention. Tissue Inhibitor of Metalloproteinases-1 (“TIMP”) is secreted by endothelial cells which also secrete MTPases. TIMP is glycosylated and has a molecular weight of 28.5 kDa. TIMP-1 regulates angiogenesis by binding to activated metalloproteinases, thereby suppressing the invasion of blood vessels into the extracellular matrix. Tissue Inhibitor of Metalloproteinases-2 (“TIMP-2”) may also be utilized to inhibit angiogenesis. Briefly, TIMP-2 is a 21 kDa nonglycosylated protein which binds to metalloproteinases in both the active and latent, proenzyme forms. Both TIMP-1 and TIMP-2 may be obtained from commercial sources such as Synergen, Boulder, Colo.

Plasminogen Activator Inhibitor-1 (PA) is a 50 kDa glycoprotein which is present in blood platelets, and can also be synthesized by endothelial cells and muscle cells. PAI-1 inhibits t-PA and urokinase plasminogen activator at the basolateral site of the endothelium, and additionally regulates the fibrinolysis process. Plasminogen Activator Inhibitor-2 (PAI-2) is generally found only in the blood under certain circumstances such as in pregnancy, and in the presence of tumors. Briefly, PAI-2 is a 56 kDa protein which is secreted by monocytes and macrophages. It is believed to regulate fibrinolytic activity, and in particular inhibits urokinase plasminogen activator and tissue plasminogen activator, thereby preventing fibrinolysis.

Lighter “d group” transition metals include, for example, vanadium, molybdenum, tungsten, titanium, niobium, and tantalum species. Such transition metal species may form transition metal complexes. Suitable complexes of the above-mentioned transition metal species include oxo transition metal complexes. Representative examples of vanadium complexes include oxo vanadium complexes such as vanadate and vanadyl complexes. Suitable vanadate complexes include metavanadate (i.e., VO₃⁻¹) and orthovanadate (i.e., VO₄³⁻) complexes such as, for example, ammonium metavanadate (i.e., NH₄VO₃), sodium metavanadate (i.e., NaVO₃), and sodium orthovanadate (i.e., Na₃VO₄). Suitable vanadyl (i.e., VO²⁺) complexes include, for example, vanadyl acetylacetonate and vanadyl sulfate including vanadyl sulfate hydrates such as vanadyl sulfate mono- and trihydrates.

Representative examples of tungsten and molybdenum complexes also include oxo complexes. Suitable oxo tungsten complexes include tungstate and tungsten oxide complexes. Suitable tungstate (i.e., WO₄²⁻) complexes include ammonium tungstate (i.e., (NH₄)₂WO₄), calcium tungstate (i.e., CaWO₄), sodium tungstate dihydrate (i.e., Na₂WO₄.2H₂O), and tungstic acid (i.e., H₂WO₄). Suitable tungsten oxides include tungsten (IV) oxide (i.e., WO₂) and tungsten (VI) oxide (i.e., WO₃). Suitable oxo molybdenum complexes include molybdate, molybdenum oxide, and molybdenyl complexes. Suitable molybdate (i.e., MoO₄²⁻) complexes include ammonium molybdate (i.e., (NH₄)₂MoO₄) and its hydrates, sodium molybdate (i.e., Na₂MoO₄) and its hydrates, and potassium molybdate (i.e., K₂MoO₄) and its hydrates. Suitable molybdenum oxides include molybdenum (VI) oxide (i.e., MoO₂), molybdenum (VI) oxide (i.e., MoO₃), and molybdic acid. Suitable molybdenyl (i.e., MoO₂²⁺) complexes include, for example, molybdenyl acetylacetonate. Other suitable tungsten and molybdenum complexes include hydroxo derivatives derived from, for example, glycerol, tartaric acid, and sugars.

A wide variety of other anti-angiogenic factors may also be utilized within the context of the present invention. Representative examples include Platelet Factor 4 (Sigma Chemical Co., #F1385); Protamine Sulphate (Clupeine) (Sigma Chemical Co., #P4505); Sulfated Chitin Derivatives (prepared from queen crab shells), (Sigma Chemical Co., #C3641; Murata et al., Cancer Res. 51:22-26, 1991); Sulfated Polysaccharide Peptidoglycan Complex (SP-PG) (the function of this compound may be enhanced by the presence of steroids such as estrogen, and tamoxifen citrate); Staurosporine (Sigma Chemical Co., #S4400); Modulators of Matrix Metabolism, including for example, proline analogs {[(L-azetidine-2-carboxylic acid (LACA) (Sigma Chemical Co., #A0760)), cishydroxyproline, d,L-3,4-dehydroproline (Sigma Chemical Co., #D0265), Thiaproline (Sigma Chemical Co., #T0631)], α,α-dipyridyl (Sigma Chemical Co., #D7505), β-aminopropionitrile fumarate (Sigma Chemical Co., #A3134)]}; MDL 27032 (4-propyl-5-(4-pyridinyl)-2(3H)-oxazolone; Merion Merrel Dow Research Institute); Methotrexate (Sigma Chemical Co., #A6770; Hirata et al., Arthritis and Rheumatism 32:1065-1073, 1989); Mitoxantrone (Polverini and Novak, Biochem. Biophys. Res. Comm. 140:901-907); Heparin (Folkman, Bio. Phar. 34:905-909, 1985; Sigma Chemical Co., #P8754); Interferons (e.g., Sigma Chemical Co., #13265); 2 Macroglobulin-serum (Sigma Chemical Co., #M7151); ChIMP-3 (Pavloff et al., J. Bio. Chem. 267:17321-17326, 1992); Chymostatin (Sigma Chemical Co., #C7268; Tomkinson et al., Biochem J. 286:475-480, 1992); β-Cyclodextrin Tetradecasulfate (Sigma Chemical Co., #C4767); Eponemycin; Camptothecin; Fumagillin (Sigma Chemical Co., #F6771; Canadian Patent No. 2,024,306; Ingber et al., Nature 348:555-557, 1990); Gold Sodium Thiomalate (“GST”; Sigma:G4022; Matsubara and Ziff, J. Clin. Invest. 79:1440-1446, 1987); (D-Penicillamine (“CDPT”; Sigma Chemical Co., #P4875 or P50OO(HCl)); P-1-anticollagenase-serum; α2-antiplasmin (Sigma Chem. Co.:A0914; Holmes et al., J. Biol. Chem. 262(4):1659-1664, 1987); Bisantrene (National Cancer Institute); Lobenzarit disodium (N-(2)-carboxyphenyl-4-chloroanthronilic acid disodium or “CCA”; Takeuchi et al., Agents Actions 36:312-316, 1992); Thalidomide; Angostatic steroid; AGM-1470; carboxynaminolmidazole; metalloproteinase inhibitors such as BB94.

Although the above anti-angiogenic factors have been provided for the purposes of illustration, it should be understood that the present invention is not so limited. In particular, although certain anti-angiogenic factors are specifically referred to above, the present invention should be understood to include analogues, derivatives and conjugates of such anti-angiogenic factors. For example, paclitaxel should be understood to refer to not only the common chemically available form of paclitaxel, but analogues (e.g., taxotere, as noted above) and paclitaxel conjugates (e.g., paclitaxel-PEG, paclitaxel-dextran, or paclitaxel-xylos).

Anti-angiogenic compositions of the present invention may additionally comprise a wide variety of compounds in addition to the anti-angiogenic factor and polymeric carrier. For example, anti-angiogenic compositions of the present invention may also, within certain embodiments of the invention, also comprise one or more antibiotics, anti-inflammatories, anti-viral agents, anti-fungal agents and/or anti-protozoal agents. Representative examples of antibiotics included within the compositions described herein include: penicillins; cephalosporins such as cefadroxil, cefazolin, cefaclor; aminoglycosides such as gentamycin and tobramycin; sulfonamides such as sulfamethoxazole; and metronidazole. Representative examples of anti-inflammatories include: steroids such as prednisone, prednisolone, hydrocortisone, adrenocorticotropic hormone, and sulfasalazine; and non-steroidal anti-inflammatory drugs (“NSAIDS”) such as aspirin, ibuprofen, naproxen, fenoprofen, indomethacin, and phenylbutazone. Representative examples of antiviral agents include acyclovir, ganciclovir, zidovudine. Representative examples of antifungal agents include: nystatin, ketoconazole, griseofulvin, flucytosine, miconazole, clotrimazole. Representative examples of antiprotozoal agents include: pentamidine isethionate, quinine, chloroquine, and mefloquine.

Within certain preferred embodiments of the invention, anti-angiogenic compositions are provided which contain one or more compounds which disrupt microtubule function. Representative examples of such compounds include paclitaxel (discussed above), estramustine (available from Sigma; Wang and Stearns Cancer Res. 48:6262-6271, 1988), epothilone, curacin-A, colchicine, methotrexate, vinblastine and 4-tert-butyl-[3-(2-chloroethyl)ureido]benzene (“tBCEU”).

Anti-angiogenic compositions of the present invention may also contain a wide variety of other compounds, including for example: α-adrenergic blocking agents, angiotensin II receptor antagonists and receptor antagonists for histamine, serotonin, endothelin; inhibitors of the sodium/hydrogen antiporter (e.g., amiloride and its derivatives); agents that modulate intracellular Ca²⁺ transport such as L-type (e.g., diltiazem, nifedipine, verapamil) or T-type Ca²⁺ channel blockers (e.g., amiloride), calmodulin antagonists (e.g., H₇) and inhibitors of the sodium/calcium antiporter (e.g., amiloride); ap-1 inhibitors (for tyrosine kinases, protein kinase C, myosin light chain kinase, Ca²⁺/calmodulin kinase II, casein kinase II); anti-depressants (e.g. amytriptyline, fluoxetine, LUVOX™ and PAXIL™); cytokine and/or growth factors, as well as their respective receptors, (e.g., the interleukins, α-, β- or γ-IFN, GM-CSF, G-CSF, epidermal growth factor, transforming growth factors alpha and beta, TNF, and antagonists of vascular epithelial growth factor, endothelial growth factor, acidic or basic fibroblast growth factors, and platelet dervived growth factor); inhibitors of the IP3 receptor (e.g., heparin); protease and collagenase inhibitors (e.g., TIMPs, discussed above); nitrovasodilators (e.g., isosorbide dinitrate); anti-mitotic agents (e.g., colchicine, anthracyclines and other antibiotics, folate antagonists and other anti-metabolites, vinca alkaloids, nitrosoureas, DNA alkylating agents, topoisomerase inhibitors, purine antagonists and analogs, pyrimidine antagonists and analogs, alkyl sulfonates); immunosuppressive agents (e.g., adrenocorticosteroids, cyclosporine); sense or antisense oligonucleotides (e.g., DNA, RNA, nucleic acid analogues (e.g., peptide nucleic acids) or any combinations of these); and inhibitors of transcription factor activity (e.g., lighter d group transition metals).

Anti-Apoptic and Neuroprotective Nucleic acids

In another preferred embodiment, the vectors express anti-apoptic or neuroprotective factors and/or are administered in conjunction with the vectors of the invention. The vector includes HRE's and a GFAP promoter.

Examples of anti-apoptic factors include: ARC, Apoptosis Repressor with CARD domain; ICAD, inhibitor of Caspase-activated DNase; CIDE, DREP-1, a Drosophila melanogaster homologue of DFF45; RICK, RIP-like interacting CLARP kinase [RICK is also referred to as CIPERK] CARD, caspase recruitment domain; CLARP, caspase-like protein with homology to caspase-8.

Anti-apoptic bioassays can be used to determine cell survival. For example, cell viability can be determined using a propidium iodide incorporation assay as described (Lee et al., 1993, J. Immunol. 151: 5208). Briefly, cells are incubated for 30 minutes with propidium iodide (125 μg/ml) at room temperature before FACS analysis with a FACScan flow cytometer (Becton-Dickinson). Under these conditions, dead cells are brightly stained while live cells are not. A minimum of 5000 cells can be counted per sample.

Neurotrophins (NT) are trophic and mitogenic proteins that have a role in the development, differentiation, connectivity, and survival of neurons in the central and peripheral nervous system, including the retina. Brain derived neurotrophic factor (BDNF), neurotrophin-3 (NT3), and neurotrophin-4 (NT4) have been demonstrated to promote retinal ganglion cell survival after injury and BDNF is thought to aid in the recovery of the retina after reattachment. NT4 (also called NT5) is the most recently discovered NT in mammals and its biological role is not fully understood. All NT knockout mice have proved lethal during early postnatal development apart from NT4 deficient mice that only show minor cellular deficits and develop normally to adulthood. NT4 knockout mice have recently been reported to require NT3 in early postnatal development and NT4 later in mature animals for survival of the sensory neurons. The biological activities of neurotrophins are mediated by two classes of cell surface receptors: the neurotrophin receptor p75, which binds all neurotrophins with similar affinity, and the trk family of receptor tyrosine kinases. Each of the three trk receptors has preferential ligands (NGF for Trk A, BDNF and NT4 for Trk B, and NT3 for Trk C.

Treatment of Related Disorders

In a preferred embodiment, diabetic retinopathy is treated with Tubedown-1 antisense to produce new vascular growth. The treatment of preproliferative diabetic retinopathy induces healing of the vascular bed, that is the development of new vessels to replace those lost, as occurs in normal wound healing in most other tissues of the body. Other antisense molecules include those of angiostatin, endostatin and the like.

In another preferred embodiment, treatment of diabetic retinopathy comprises diabetic subjects who have experienced significant loss of vessels in preproliferative diabetic retinopathy, and treating said patients with the vector delivering a hypoxia-regulated, GFAP promoter linked to antisense Tubedown-1, to reduce expression of Tubedown-1 exclusively in the hypoxia regions where vessels have been lost to encourage revascularization of the affected area.

In another preferred embodiment, treatment of diabetic retinopathy comprises identifying diabetic subjects who have experienced significant loss of vessels in preproliferative diabetic retinopathy and treating said patients with a vector comprising a GFAP promoter linked to antisense for Tubedown-1, and applying low-level laser energy to the regions of capillary loss to locally activate antisense for Tubedown-1 and promote revascularization of the affected area.

Inhibition of Gene Expression: in a preferred embodiment, the invention provides methods for treating cells comprising an antisense Tubedown-1 nucleic acid molecule. Such treatment methods comprise administering an antisense oligonucleotide to cells that comprise an oligonucleotide sequence of Tubedown-1.

This relationship between an oligonucleotide and its complementary nucleic acid target to which it hybridizes is commonly referred to as “antisense.” “Targeting” refers to oligonucleotides specific for a chosen nucleic acid target. The process usually begins with identifying a nucleic acid sequence whose function is to be modulated, such as for example, a Tubedown-1. The targeting process also includes determination of a site or sites within the nucleic acid sequence for the oligonucleotide interaction to occur such that the desired effect—modulation of gene expression—will result. Once the target site or sites have been identified, oligonucleotides are chosen which are sufficiently complementary to the target, i.e., hybridize sufficiently well and with sufficient specificity, to give the desired modulation.

One way to interfere with gene function is to introduce the interference nucleic acids (RNA, DNA, or modified DNA) into cells. The sequence of the interference molecule is complementary to that of an RNA molecule normally transcribed by the cell. Binding of the interference molecule to the endogenous target RNA can inhibit expression of the target in any one of several ways, e.g., by preventing ribosome binding and thus interfering with translation.

Inhibition of gene expression may be quantified by measuring either the endogenous target RNA or the protein produced by translation of the target RNA. Techniques for quantifying RNA and proteins are well known to one of ordinary skill in the art. In certain preferred embodiments gene expression is inhibited by at least 10%, preferably by at least 33%, more preferably by at least 50%, and yet more preferably by at least 80%. In particularly preferred embodiments of the invention gene expression is inhibited by at least 90%, more preferably by at least 95%, or by at least 99% up to 100% within cells in the organism. In preferred embodiments of the invention inhibition occurs rapidly after the antisense oligonucleotide organism comes into contact with the Tubedown-1 nucleic acid. In preferred embodiments significant inhibition of gene expression occurs within 24 hours after administration of the antisense nucleic acid. In more preferred embodiments significant inhibition occurs within 12 hours after administration of the antisense nucleic acid. In yet more preferred embodiments significant inhibition occurs between about 6 to 12 hours after administration of the antisense nucleic acid. In yet more preferred embodiments significant inhibition occurs within less than about 6 hours after administration of the antisense nucleic acid. By significant inhibition is meant sufficient inhibition to result in a detectable phenotype (e.g., inhibition of Tubedown-1) or a detectable decrease in RNA and/or protein corresponding to the gene being inhibited.

Other methods of inhibiting gene expression is use of interference RNA. The term “interference RNA (RNAi)” as used herein refers to a polyribonucleotide structure formed by either a single self-complementary RNA strand or by at least two complementary RNA strands, for example, RNA, having a complementary base sequence to the base sequence of a messenger RNA (mRNA). The degree of complementary need not necessarily be 100 percent. Rather, it must be sufficient to allow the formation of a double-stranded structure under the conditions employed. The term “complementary” as used herein refers to a nucleotide sequence that is related to another nucleotide sequence by the Watson-Crick base-pairing rules, i.e., the sequence A-T-G-C in a DNA strand is complementary to the sequence T-A-C-G in a second DNA strand and to the sequence U-A-C-G in an RNA strand. The term “quantifying” when used in the context of quantifying transcription levels of a gene can refer to absolute or to relative quantification. Absolute quantification may be accomplished by inclusion of known concentration(s) of one or more target nucleic acids (e.g. control nucleic acids such as Bio B or with known amounts the target nucleic acids themselves) and referencing the hybridization intensity of unknowns with the known target nucleic acids (e.g. through generation of a standard curve). Alternatively, relative quantification can be accomplished by comparison of hybridization signals between two or more genes, or between two or more treatments to quantify the changes in hybridization intensity and, by implication, transcription level.

The “percentage of sequence identity” or “sequence identity” is determined by comparing two optimally aligned sequences or subsequences over a comparison window or span, wherein the portion of the polynucleotide sequence in the comparison window may optionally comprise additions or deletions (i.e., gaps) as compared to the reference sequence (which does not comprise additions or deletions) for optimal alignment of the two sequences. The percentage is calculated by determining the number of positions at which the identical subunit (e.g. nucleic acid base or amino acid residue) occurs in both sequences to yield the number of matched positions, dividing the number of matched positions by the total number of positions in the window of comparison and multiplying the result by 100 to yield the percentage of sequence identity. Percentage sequence identity when calculated using the programs GAP or BESTFIT and is calculated using default gap weights.

In order to achieve inhibition of a target gene, e.g. Tubedown-1 selectively within a given subject which it is desired to control, an RNAi preferably exhibits a low degree of sequence identity with other unrelated genes in the subject. Preferably the degree of identity is less than approximately 80%. Still more preferably the degree of identity is less than approximately 70%. Yet more preferably the degree of identity is less than approximately 50%. Untranslated regions (UTRs), i.e., 5′ and 3′ UTRs, frequently display a low degree of conservation across species since they are not constrained by the necessity of coding for a fimctional protein. Thus, in certain preferred embodiments the gene portion comprises or includes a UTR.

Selection of appropriate RNAi is facilitated by using computer programs that automatically align nucleic acid sequences and indicate regions of identity or homology. Such programs are used to compare nucleic acid sequences obtained, for example, by searching databases such as GenBank or by sequencing PCR products. Comparison of nucleic acid sequences from a number of variants found in a species allows the selection of nucleic acid sequences that display an appropriate degree of identity within the species, e.g. human. Southern blots are performed to allow a determination of the degree of identity between genes in target species. By performing Southern blots at varying degrees of stringency, as is well known in the art, it is possible to obtain an approximate measure of identity. These procedures allow the selection of RNAi that exhibit a high degree of complementarity to target nucleic acid sequences in a subject to be controlled. One skilled in the art will realize that there is considerable latitude in selecting appropriate regions of genes for use in the present invention.

In a preferred embodiment, small interfering RNA (siRNA) either as RNA itself or as DNA, is delivered to a cell using an expression plasmid or virus and the coding sequence for small hairpin RNAs that are processed to siRNAs.

In another preferred embodiment, a DNA cassette for the cloning of small hairpin sequences permit their expression and processing using RNA polymerase II. This system enables efficient transport of the pre-siRNAs to the cytoplasm where they are active and permit the use of regulated and tissue specific promoters for gene expression.

In another preferred embodiment, cassette comprises hammerhead and hairpin ribozymes flanking the cloning sites for hairpin RNA of variable sequence, depending on the target of RNA interference.

In another preferred embodiment, cloning site for insertion of the hairpin RNA into an expression vector can be in any nucleotide location, as internal base-pairing is preserved in the flanking hammerhead ribozyme (Rz) and hairpin ribozyme.

Preferred siRNA's of the invention will hybridize (bind) to a target sequence, particularly a target oligonucleotide of Tubedown-1, in this instance of the invention, for treatment of preproliferative diabetic retinopathy and modulate the expression of Tubedown-1. The invention may be used against protein coding gene products as well as non-protein coding gene products. Examples of non-protein coding gene products include gene products that encode ribosomal RNAs, transfer RNAs, small nuclear RNAs, small cytoplasmic RNAs, telomerase RNA, RNA molecules involved in DNA replication, chromosomal rearrangement and the like.

In the context of this invention “modulation” means either inhibition or stimulation. Inhibition of Tubedown-1 expression, in this instance of the invention, for treatment of preproliferative diabetic retinopathy is presently the preferred form of modulation. Preferred methods for measuring the modulation of Tubedown-1, are routine in the art, for example by Northern blot assay of MRNA expression or Western blot assay of protein expression and as taught in the examples of the instant application. “Hybridization”, in the context of this invention, means hydrogen bonding, also known as Watson-Crick base pairing, between complementary bases, usually on opposite nucleic acid strands or two regions of a nucleic acid strand. Guanine and cytosine are examples of complementary bases which are known to form three hydrogen bonds between them. Adenine and thymine are examples of complementary bases which form two hydrogen bonds between them. “Specifically hybridizable” or “specific for” and “complementary” are terms which are used to indicate a sufficient degree of complementarity such that stable and specific binding occurs between the DNA or RNA target and the oligonucleotide. It is understood that an oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

In preferred embodiments of this invention, oligonucleotides which are targeted to mRNA encoding Tubedown-1, and suitable oligonucleotides can be derived for example, from cloned Tubedown-1 . In accordance with this invention, persons of ordinary skill in the art will understand that MRNA includes not only the coding region which carries the information to encode a protein using the three letter genetic code, including the translation start and stop codons, but also associated ribonucleotides which form a region known to such persons as the 5′-untranslated region, the 3′-untranslated region, the 5′ cap region, intron regions and intron/exon or splice junction ribonucleotides. Thus, oligonucleotides may be formulated in accordance with this invention which are targeted wholly or in part to these associated ribonucleotides as well as to the coding ribonucleotides. In preferred embodiments, the oligonucleotide is targeted to a translation initiation site (AUG codon) or sequences in the coding region, 5′ untranslated region or 3′-untranslated region of the Tubedown-1 mRNA. The functions of messenger RNA to be interfered with include all vital fuictions such as translocation of the RNA to the site for protein translation, actual translation of protein from the RNA, splicing or maturation of the RNA and possibly even independent catalytic activity which may be engaged in by the RNA. The overall effect of such interference with the RNA function is to cause interference with Tubedown-1 protein expression.

Certain preferred oligonucleotides of this invention are chimeric oligonucleotides. “Chimeric oligonucleotides” or “chimeras”, in the context of this invention, are oligonucleotides which contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the RNA target) and a region that is a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of antisense inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art. In one preferred embodiment, a chimeric oligonucleotide comprises at least one region modified to increase target binding affinity, and, usually, a region that acts as a substrate for RNAse H. Affinity of an oligonucleotide for its target (in this case, a nucleic acid encoding ras) is routinely determined by measuring the Tₘ of an oligonucleotide/target pair, which is the temperature at which the oligonucleotide and target dissociate; dissociation is detected spectrophotometrically. The higher the Tₘ the greater the affinity of the oligonucleotide for the target. In a more preferred embodiment, the region of the oligonucleotide which is modified to increase Tubedown-1 mRNA binding affinity comprises at least one nucleotide modified at the 2′ position of the sugar, most preferably a 2′-O-alkyl, 2′-O-alkyl-O-alkyl or 2′-fluoro-modified nucleotide. Such modifications are routinely incorporated into oligonucleotides and these oligonucleotides have been shown to have a higher Tₘ (i.e., higher target binding affinity) than; 2′-deoxyoligonucleotides against a given target. The effect of such increased affinity is to greatly enhance RNAi oligonucleotide inhibition of Tubedown-1 gene expression. RNAse H is a cellular endonuclease that cleaves the RNA strand of RNA:DNA duplexes; activation of this enzyme therefore results in cleavage of the RNA target, and thus can greatly enhance the efficiency of RNAi inhibition. Cleavage of the RNA target can be routinely demonstrated by gel electrophoresis. In another preferred embodiment, the chimeric oligonucleotide is also modified to enhance nuclease resistance. Cells contain a variety of exo- and endo-nucleases which can degrade nucleic acids. A number of nucleotide and nucleoside modifications have been shown to make the oligonucleotide into which they are incorporated more resistant to nuclease digestion than the native oligodeoxynucleotide. Nuclease resistance is routinely measured by incubating oligonucleotides with cellular extracts or isolated nuclease solutions and measuring the extent of intact oligonucleotide remaining over time, usually by gel electrophoresis. Oligonucleotides which have been modified to enhance their nuclease resistance survive intact for a longer time than unmodified oligonucleotides. A variety of oligonucleotide modifications have been demonstrated to enhance or confer nuclease resistance. Oligonucleotides which contain at least one phosphorothioate modification are presently more preferred. In some cases, oligonucleotide modifications which enhance target binding affinity are also, independently, able to enhance nuclease resistance. Some desirable modifications can be found in De Mesmaeker et al. Acc. Chem. Res. 1995, 28:366-374.

Specific examples of some preferred oligonucleotides envisioned for this invention include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NH—O—CH₂, CH,—N(CH₃)—O—CH₂ [known as a methylene(methylimino) or MMI backbone], CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH,). The amide backbones disclosed by De Mesmaeker et al. Acc. Chem. Res. 1995, 28:366-374) are also preferred. Also preferred are oligonucleotides having morpholino backbone structures (Summerton and Weller, U.S. Pat. No. 5,034,506). In other preferred embodiments, such as the peptide nucleic acid (PNA) backbone, the phosphodiester backbone of the oligonucleotide is replaced with a polyamide backbone, the nucleobases being bound directly or indirectly to the aza nitrogen atoms of the polyamide backbone (Nielsen et al. Science 1991, 254, 1497). Oligonucleotides may also comprise one or more substituted sugar moieties. Preferred oligonucleotides comprise one of the following at the 2′ position: OH, SH, SCH₃, F, OCN, OCH₃OCH₃, OCH₃O(CH₂)ₙCH₃, O(CH₂)ₙNH₂ or O(CH₂)ₙCH₃ where n is from 1 to about 10; C₁ to C₁₀ lower alkyl, alkoxyalkoxy, substituted lower alkyl, alkaryl or aralkyl; Cl; Br; CN; CF₃; OCF₃; O—, S—, or N-alkyl; O—, S—, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-O—CH₂CH₂OCH₃, also known as 2′-O-(2-methoxyethyl)] (Martin et al., Helv. Chim. Acta, 1995, 78, 486). Other preferred modifications include 2′-methoxy (2′-O—CH₃), 2′-propoxy (2′-OCH₂CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Oligonucleotides may also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N₆ (6-aminohexyl)adenine and 2,6-diaminopurine. Komberg, A., DNA Replication, W. H. Freeman & Co., San Francisco, 1980, pp75-77; Gebeyehu, G., et al. Nucl. Acids Res. 1987, 15:4513). A “universal” base known in the art, e.g., inosine, may be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278) and are presently preferred base substitutions.

Another modification of the oligonucleotides of the invention involves chemically linking to the oligonucleotide one or more moieties or conjugates which enhance the activity or cellular uptake of the oligonucleotide. Such moieties include but are not limited to lipid moieties such as a cholesterol moiety, a cholesteryl moiety (Letsinger et al., Proc. Natl. Acad. Sci. USA 1989, 86, 6553), cholic acid (Manoharan et al. Bioorg. Med. Chem. Let. 1994, 4, 1053), a thioether, e.g., hexyl-S-tritylthiol (Manoharan et al. Ann. N.Y Acad. Sci. 1992, 660, 306; Manoharan et al. Bioorg. Med. Chem. Let. 1993, 3, 2765), a thiocholesterol (Oberhauser et al., Nucl. Acids Res. 1992, 20, 533), an aliphatic chain, e.g., dodecandiol or undecyl residues (Saison-Behmoaras et al. EMBO J. 1991, 10, 111; Kabanov et al. FEBS Lett. 1990, 259, 327; Svinarchuk et al. Biochimie 1993, 75, 49), a phospholipid, e.g., di-hexadecyl-rac-glycerol or triethylammonium 1,2-di-O-hexadecyl-rac-glycero-3-H-phosphonate (Manoharan et al. Tetrahedron Lett. 1995, 36, 3651; Shea et al. Nucl. Acids Res. 1990, 18, 3777), a polyamine or a polyethylene glycol chain (Manoharan et al. Nucleosides & Nucleotides 1995, 14, 969), or adamantane acetic acid (Manoharan et al. Tetrahedron Lett. 1995, 36, 3651). Oligonucleotides comprising lipophilic moieties, and methods for preparing such oligonucleotides are known in the art, for example, U.S. Pat. Nos. 5,138,045, 5,218,105 and 5,459,255.

It is not necessary for all positions in a given oligonucleotide to be uniformly modified, and in fact more than one of the aforementioned modifications may be incorporated in a single oligonucleotide or even at within a single nucleoside within an oligonucleotide. The present invention also includes oligonucleotides which are chimeric oligonucleotides as hereinbefore defined.

The oligonucleotides used in accordance with this invention may be conveniently and routinely made through the well-known technique of solid phase synthesis. Equipment for such synthesis is sold by several vendors including Applied Biosystems. Any other means for such synthesis may also be employed; the actual synthesis of the oligonucleotides is well within the talents of one of ordinary skill in the art. It is also well known to use similar techniques to prepare other oligonucleotides such as the phosphorothioates and alkylated derivatives. It is also well known to use similar techniques and commercially available modified amidites and controlled-pore glass (CPG) products such as biotin, fluorescein, acridine or psoralen-modified amidites and/or CPG (available from Glen Research, Sterling VA) to synthesize fluorescently labeled, biotinylated or other modified oligonucleotides such as cholesterol-modified oligonucleotides.

The oligonucleotides in accordance with this invention preferably comprise from about 8 to about 50 nucleic acid base units. In the context of this invention it is understood that this encompasses non-naturally occurring oligomers as hereinbefore described, having about 8 to about 50 monomers.

Sequence Alignments

Methods of alignment of sequences for comparison and to identify previously unidentified sequences, are well known in the art. Optimal alignment of sequences for comparison may be conducted by the local homology algorithm of Smith and Waterman, Adv. Appl. Math. 2: 482 (1981), by the homology alignment algorithm of Needleman and Wunsch J. Mol. Biol. 48: 443 (1970), by the search for similarity method of Pearson and Lipman, Proc. Natl. Acad. Sci USA 85: 2444 (1988), by computerized implementations of these algorithms (including, but not limited to CLUSTAL in the PC/Gene program by Intelligenetics, Moutain View, Calif., GAP, BESTFIT, FASTA, and TFASTA in the Wisconsin Genetics Software Package, Genetics Computer Group (GCG), 575 Science Dr., Madison, Wis., USA), or by inspection. In particular, methods for aligning sequences using the CLUSTAL program are well described by Higgins and Sharp in Gene, 73: 237-244 (1988) and in CABIOS 5: 151-1.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in pertinent part for all purposes to the same extent as if each individual publication or patent document were so individually denoted. By their citation of various references in this document, Applicants do not admit any particular reference is “prior art” to their invention.

### EXAMPLES

The following examples serve to illustrate the invention without limiting it thereby. It will be understood that variations and modifications can be made without departing from the spirit and scope of the invention.

Materials and Methods

Cell specificity, selectivity, and regulation by hypoxia in RPE, Muller cells and in retinal explants: rAAV vectors containing HRE-mediated GFP driven by the Rpe65 or GFAP promoter giving RPE- or Muller cell-specific expression, respectively, are added to the triplicate cultures maintained in 2, 5, 10 and 20% oxygen for the following 7 days. Hypoxia-regulated expression is indicated by a higher percentage of more intensely stained cells at lower oxygen concentrations, and absence of stain in 10 and 20% oxygen. Specificity in vitro is determined by transfection of a mixture of Muller and primary mouse RPE cells and/or ARPE-19 cells, and determination of the percent of cells that express both GFP and a Muller-cell specific marker (GFAP), and the percent that express GFP and RPE-specific cytokeratin. Specificity in retinal explants transfected with rAAV -HRE-GFP constructs with a GFAP promoter (rAAV-HRE-GFAP-GFP) are evaluated by determining whether any GFP is detected that does not co-localize with GFAP in Muller cells. Selective expression in ischemic areas of retina are evaluated in retinal explants transfected with HRE-regulated Muller specific vector and cultured for 24 hours at various oxygen tensions. Image analysis of flat mounts are compared to the distribution of the HRE-regulated expression of GFP to the distribution of retinal vessels labeled with Cy3 labeled isolectin B4.

Efficacy of transfecting Muller-cell specific vectors bearing carrying endostatin, angiostatin, or Tubedown-1 genes: mice are given intravitreal injections of vectors 24 hours before placement in hyperoxia for 5 days. Neovascular scores are determined after 7 days in room air. 32 animals are anaesthetized and given bilateral intravitreal injections on P6 with rAAV vectors expressing GFP, and eyes fixed on P19. Specificity of expression in Muller cells is examined by confocal microscopy in retinal sections of left eyes counter stained with Cy-3 (red) labeled antibodies to GFAP. Localization of GFP is compared with that of Muller cell markers. To examine selective of expression in hypoxic (non-perfused) regions of the retina, retinal vessels in flat-mounted right eyes are displayed by labeling vascular endothelial cells with Cy3 labeled isolectin B4, specific for endothelial cells. The mean distance separating red and green fluorescence (determined in confocal images) verifies gene expression in avascular, hypoxic regions of the retina and provide an index for the severity of retinal damage that elicits expression.

Inhibition of retinal neovascularization with genes expressed in the RPE cell and Muller cell. NV in animals are injected subretinally with AAV-HRE-Rpe65-SpAm2* vectors targeting RPE expression or intravitreally with AAV-HRE-GFAP-SpAm2* vectors for Muller-cell expression. Three replica experiments—each with 32 pups and 4 mothers—are conducted with each cell-specific vector. In each experiment, half of the pups are injected with rAAV-HRE-(Rpe65 or GFAP)-SpAm2*, and the other half with rAAV-(Rpe65 or GFAP)-HRE-GFP. Left eyes are treated and right eyes serve as controls. NV scores are determined in 8-15 paraffin sections of both eyes by counting the number of vascular endothelial cell nuclei in the vitreous. Mean NV scores in the GFP, SpAm2*, and contralateral control eyes (N=48 in each group) are statistically compared. Bilateral injection of another anti-angiogenic substance has been approved by the IACUC.

### Example 1

### Production of rAAV Vectors Containing GFAP Promoter and HRE Element

Production of vectors. Expression plasmids containing muscle-specific promoters and HRE elements linked to a reporter gene have been previously described (Prentice, H., et al. 1997 Cardiovasc Res, 35:567-74; Webster, K. A., Kubasiak, L. A., Prentice, H. and Bishopric, N. H.: Stable germline transmission of a hypoxia-activated molecular gene switch. From the double helix to molecular medicine, (ed.W.J. Whelan et al.), Oxford University Press, (2003). Using adaptations of these methods, adeno-associated virus (rAAV) vectors containing HRE elements are constructed in rAAV 2/1, selected for its efficient transduction of retinal cells and rapid onset of expression (Auricchio, A. et al. 2002 Mol Ther 6:490-4).

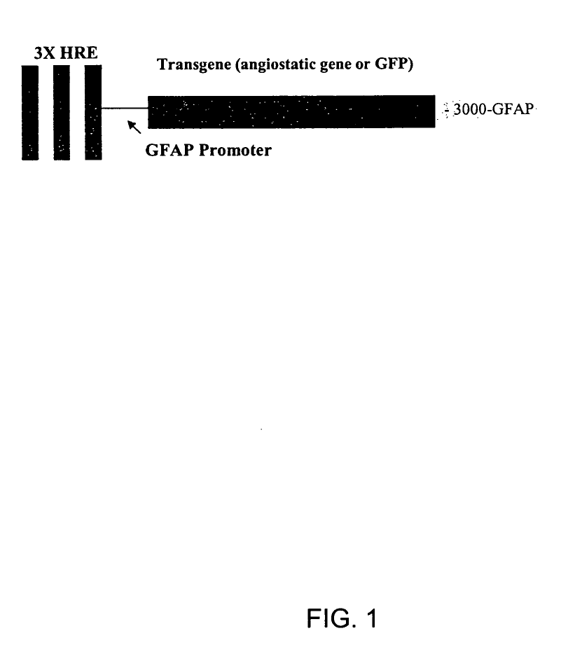

The design of an exemplary vector is shown in FIG. 1. The HRE in the vector activates the promoter for GFAP, driving expression of an anti-angiogenic gene product or a reporter gene (for example, green fluorescent protein, GFP) in hypoxic cells that express GFAP. The design of the vectors is such that the anti-angiogenic factor, for example, endostatin, is specifically expressed in GFAP-expressing cells, specifically in response to hypoxia.

In developing the constructs of the invention, it was previously shown that placing multiple copies of an HRE upstream of a reporter gene had only small effects on gene expression in room air, but resulted in 5 to 6 fold increase in reporter (luciferase) expression when transfected cultures were placed in hypoxia (8-12 mm Hg). Luciferase expression in transfected cardiac muscle increased 5-fold one hour after ligation of an artery, and remained similarly elevated for 4 hours after the heart was reperfused. By contrast, constructs lacking an HRE exhibited low levels of constitutive expression, and no response to hypoxia (Prentice, H. et al. 1997 Cardiovasc Res 35:567-74). Previous studies utilizing VEGF as the gene of interest in a heart model revealed that addition of a HRE to rAAV-VEGF vectors supported an even greater response to hypoxia, i.e., VEGF expression increased 16-fold in hearts transfected with rAAV-VEGF, and 199-fold in hearts transfected with rAAV-HRE(9)-VEGF vector containing a concatamer of 9 HRE consensus sequences (Su, H., J. Arakawa-Hoyt, and Y.W. Kan, 2002. Proc Natl Acad Sci USA, 99: 9480-5.

The rAAV particles of the invention can be propagated and purified by techniques known in the art, for example using tools developed by the inventors, such as a fast throughput column chromatography method for isolation of rAAV in high yield and high purity (Anderson, R. et al., 2000 J Virol Methods 85:23-34), following production in helper plasmids and cell lines. An exemplary cell line for this purpose is a cell line that eliminates production of wild-type adenovirus by splitting the rep and cap genes (Whiteway, A. et al. 2003 J Virol Methods 114:1-10). The foregoing plasmids advantageously include a selection mechanism that facilitates positive selection of the cell lines producing the highest quantities of recombinant rAAV.

The promoter in the construct can incorporate a HRE silencer component as previously described, in combination with a minimal tissue-specific domain of the GFAP promoter. The promoter constructs are readily incorporated into adeno-associated virus vectors as described above and have been shown to retain their cell specific properties, i.e., normoxic silencing and hypoxia-inducible characteristics (several hundred fold inducibility) in cell culture and in vivo (Webster, K. A., Kubasiak, L. A., Prentice, H. and Bishopric, N. H.: Stable germline transmission of a hypoxia-activated molecular gene switch. From the double helix to molecular medicine, (ed. W. J. Whelan et al.), Oxford University Press, (2003).

### Example 2

### In Vitro Protocols for Testing Gene Therapy Vectors

This example describes a method of in vitro testing of rAAV vectors of the invention for their efficacy in gene therapy for the retina.

An organ culture method using retinal explants is used according to a modified protocol as described (Ogilivie J M et al., J. Neurosci Methods 87:57-65, 1999). Eyes from neonatal mice are enucleated and transferred to Dulbecco's Modified Eagle Medium (DMEM) containing 10% fetal calf serum (FCS). The retina isolated from the eye and transferred onto a Millicell-CM culture insert with fresh DMEM sufficient to barely cover the explant. The retina is cut at the 3, 6, 9, and 12 o'clock positions to flatten the whole mount, and placed photoreceptor-side down onto the center of the insert. Inserts are placed in six-well tissue culture plates containing 1 ml of DMEM containing 10% FCS plus Fungizone (1.25 μg/ml), with or without the specific vector.

To test the response of vectors containing HRE elements to hypoxia, vectors are added to triplicate cultures of explants or of cells (such as Muller cells and a control cell type, or mixed cultures thereof) maintained in 2, 5, 10 and 20% oxygen for 7 days. The transgene expressed in vectors used for this purpose can be a marker protein such as green fluorescent protein (GFP) which is detected as a fluorescent signal, for example by fluorescence microscopy, in transduced cells expressing the transgene. Hypoxia-regulated expression is indicated by a higher percentage of more intensely fluorescent cells at lower oxygen concentrations, and absence of fluorescence in normoxia (about 10-20% oxygen).

In retinal explants, specificity of expression in Muller cells is tested by comparing results of explants transfected with rAAV constructs including a GFAP promoter (i.e., rAAV-HRE-GFAP-GFP) with control constructs without a GFAP promoter (i.e., rAAV-HRE-GFP). Image analysis of flat mounts is used to compare the distribution of the HRE-regulated expression of GFP to the distribution of retinal vessels labeled with Cy3 labeled isolectin B4, as described (Haigh, J. J. et al. 2003 Dev Biol 262:225-41; Coffin, J. D. et al. 1991Dev Biol 148:51-62; Albini, A., et al.1996 Nature Medicine 2:1371-75). Muller and glial cell specificity of expression is indicated by absence of GFP fluorescence in all cells except Muller cells and retinal glial cells, confirmed by co-localization of GFP and GFAP in both cell types, for example as described (Sarthy V P et al., Invest. Ophthalmol. Vis. Sci. 39:212-216, 1998; Cho E. Y et al., Histochem J. 34:, 589-600, 2002).

### Example 3

### Animal Models of Retinal Neovascularization

This example describes several animal models useful for testing the efficacy of the vectors in inhibiting various forms of retinal angiogeneis in vivo. Murine model of proliferative retinopathy. Exposure of neonatal (7 day-old) rats and mice to hyperoxia for 5 days causes vaso-obliteration and extensive vessel loss, resulting in an exacerbated model of capillary dropout in diabetes. Upon return to room air, the non-perfused, hypoxic retinal regions begin to produce VEGF. By 7 days, neovascularization occurs in 100% of the mice. The extent of non-perfused retina modulates both the risk for and the severity of neovascularization in rodent models and in patients (Smith, L. E., et al., 1994. Invest Ophthalmol Vis Sci., 35:101-11; Reynaud, X. and C. K. Dorey 1994 Invest Ophthalmol Vis Sci. 35:3169-77; Dorey, C. K., et al. 1996 Arch Ophthalmol 114:1210-7).

Laser-induced choroidal neovascularization model. Aged C57 B1/6J mice, (approximately 16 months) are treated with laser to induce choroidal neovascularization as described (Espinosa-Heidmann, D. G., et al. 2002 Invest Ophthalmol Vis Sci. 43:1567-73). The disruption of the choriocapillaris caused by placing several small laser burns around the optic nerve in the mouse eye results in increased expression of VEGF, evidence of choroidal hypoxia.

### Other Embodiments

While the above specification contains many specifics, these should not be construed as limitations on the scope of the invention, but rather as examples of preferred embodiments thereof. Many other variations are possible. Accordingly, the scope of the invention should be determined not by the embodiments illustrated, but by the appended claims and their legal equivalents.

1. A purified nucleic acid construct comprising:
at least one nucleic acid encoding an anti-angiogenic protein, operably linked to a GFAP promoter.

2. The nucleic acid construct of claim 1, further comprising at least one of a hypoxia regulated element, an enhancer element and a silencer element.

3. The nucleic acid construct of claim 1, wherein said anti-angiogenic protein is selected from the group consisting of endostatin, angiostatin and Tubedown-1.

4. A vector comprising:
a purified nucleic acid construct comprising:
at least one nucleic acid encoding an anti-angiogenic protein operably linked to a GFAP promoter.

5. The vector of claim 4, wherein said anti-angiogenic protein is selected from the group consisting of endostatin, angiostatin and Tubedown-1.

6. The vector of claim 4, wherein said vector is an expression vector.

7. The vector of claim 6, wherein said vector is a plasmid.

8. The vector of claim 6, wherein said vector is a viral vector selected from the group consisting of an adenoviral vector, a rAAV vector and a lentiviral vector.

9. The vector of claim 4, wherein the GFAP promoter is up-regulated by irradiating tissue with a laser beam, light, radiation, Cystatin C, transpupillary thermography in an amount sufficient to upregulate GFAP promoter driven expression of a nucleic acid under said promoters control.

10. The vector of claim 9, wherein the amount of light energy sufficient to upregulate GFAP promoter driven expression of a nucleic acid under said promoters control is a wavelength of light energy is from about 10 nm up to 2000 nn.

11. The vector of claim 9, wherein upregulation of the GFAP promoter upregulates expression of the anti-angiogenic factor as compared to a normal tissue and a tissue comprising said vector.

12. The vector of claim 9, wherein tissue specific expression by the vector is regulated by regulated by irradiating tissue comprising the vector with a laser beam, light, radiation, Cystatin C, transpupillary thermography in an amount sufficient to upregulate GFAP promoter driven expression of a nucleic acid under said promoters control.

13. A cell transduced with a vector, the vector comprising:
at least one nucleic acid encoding an anti-angiogenic protein, operably linked to a GFAP promoter.

14. The vector of claim 13, further comprising at least one of a hypoxia regulated element, an enhancer element and a silencer element.

15. The vector of claim 13, wherein said anti-angiogenic protein is selected from the group consisting of endostatin, angiostatin and Tubedown-1.

16. The vector of claim 13, wherein the GFAP promoter is up-regulated by targeting a laser to transduced tissue.

17. The vector of claim 16, wherein upregulation of the GFAP promoter upregulates expression of the anti-angiogenic factor as compared to a normal tissue and a tissue comprising said vector.

18. The vector of claim 13, wherein tissue specific expression by the vector is regulated by irradiating tissue comprising the vector with a laser beam, light, radiation, Cystatin C, transpupillary thermography in an amount sufficient to upregulate GFAP promoter driven expression of a nucleic acid under said promoters control.

19. A cell transduced with a vector, the vector comprising:
a purified nucleic acid construct comprising
at least one nucleic acid encoding an anti-angiogenic protein operably linked to a GFAP promoter.

20. The cell of claim 19, wherein said anti-angiogenic protein is selected from the group consisting of endostatin, angiostatin and Tubedown-1.

21. The cell of claim 19, wherein said vector is an expression vector.

22. The cell of claim 19, wherein said vector is a plasmid.

23. The cell of claim 19, wherein said vector is a viral vector selected from the group consisting of an adenoviral vector, a rAAV vector and a lentiviral vector.

24. The cell of claim 19, wherein the GFAP promoter is up-regulated by targeting a laser to transduced tissue.

25. The cell of claim 24, wherein upregulation of the GFAP promoter upregulates expression of the anti-angiogenic factor in the cell as compared to a normal cell and a cell comprising said vector.

26. The cell of claim 19, wherein cell specific expression by the vector is regulated by irradiating tissue with a laser beam, light, radiation, Cystatin C, transpupillary thermography in an amount sufficient to upregulate GFAP promoter driven expression of a nucleic acid under said promoters control.

27. A method of preventing, reducing or delaying neovascularization, the method comprising the steps of:
(a) providing a subject having or at risk of developing neovascularization in a tissue; and
(b) transducing at least one GFAP-expressing cell type of said subject with an expression vector comprising a purified nucleic acid construct comprising:
at least one nucleic acid encoding an anti-angiogenic protein operably linked to a GFAP promoter, wherein expression of said anti-angiogenic protein by said transduced cell prevents, reduces or delays neovascularization in said tissue in said subject.

28. The method of claim 27, wherein said tissue is an ocular tissue selected from at least one of the group consisting of retina, vitreous and choroid.

29. The method of claim 27, wherein said anti-angiogenic protein is selected from the group consisting of angiostatin, endostatin and Tubedown-1.

30. The method of claim 27, wherein said expression vector further comprises a hypoxia-regulated element, and the expression of said anti-angiogenic protein by said cell is increased under hypoxic conditions.

31. The method of claim 27, further comprising the step of:
(c) irradiating said tissue with a laser beam sufficient to upregulate GFAP promoter expression in said GFAP-expressing cell type, wherein the level of expression of said anti-anti-angiogenic protein driven by said GFAP promoter is increased in an amount sufficient to prevent, reduce or delay neovascularization in said tissue.

32. A method of preventing, reducing or delaying neovascularization in an eye, the method comprising the steps of:
(a) providing a subject having or at risk of developing a condition involving neovascularization in a compartment of at least one eye; and
(b) transducing at least one GFAP-expressing cell type in said eye of said subject with an expression vector comprising at least one nucleic acid encoding an anti-angiogenic protein operably linked to a GFAP promoter, wherein expression of said anti-angiogenic protein by said transduced cell prevents, reduces or delays neovascularization in said eye of said subject.

33. The method of claim 32, wherein said GFAP-expressing cell type is a Muller cell or a glial cell of the retina.

34. The method of claim 32, further comprising the step of:
(c) irradiating tissue with a laser beam, light, radiation, Cystatin C, transpupillary thermography in an amount sufficient to upregulate GFAP promoter driven expression of a nucleic acid under said promoters control.
